SEQUENCE LISTING (24 sequences)

SEQ ID NO: 1 - DNA, 1001 bp, Camellia sinensis
  source 1..1001: mol_type = genomic DNA
gaaggctctggagtagctgaagttgttatgagcttgtctaggccgaaatcagcgaggtgagcttcaaaatcggcgtcgaataggacgttctgaggcttgacatcgccatgaaccatggcggtggagtggaggaaggcgaggccgcgggcgattccgagggctattaggtggcgcattggccaattcaatacatgcccgtcttggtgagaagcttcttgaagcaatgtggctaggtttccgttaggcatatagtcgtagactaagagtctgaggtctggtggtccggcgaagtacccacggaggactgtgaggtttctgtgcttcactctcccgagcgattcggcttcttttctgaacatgttttcgtctagcgatccatcagggagtctccgaatcgaaagcaccattccatcactgtaacaggctttgaagactaacccgtatcgagtcctgcttagaacgttctcttcatcgaattgtctcgttgcttcggttgtttcngctagagtgatcttgttattgaacataacaagctttggaccgccattatcgccacttccacgacctccgctggctgcagctgagcttgctcttgctgggctgcgctttttctctccggcagccttttctttgagcctcttgcgccaccgcaagagactgtaagtgtagaagcaacaacacagtgctaagaggaaaccaccactaacagccatggcaataaacatgatcagcctcttcttcctattactcatctcttcgcatttcgtgcttaagggtttcccacataagttcggatttcctgcataatcagatggatcgttgaatcttgaagccagcattgttggaatctcgccggagaggttgttttgggatacattgaagtagaccaagctagagatgagtgaaatgtttgctggaatcggtccggtcaggttgtttgcagagagattgaggactgtgaggtttgataaattggacaatgagtctggtatttggcctgg

SEQ ID NO: 2 - DNA, 20 bp, Camellia sinensis
  source 1..20: mol_type = other DNA
gaagactaacccgtatcgag

SEQ ID NO: 3 - DNA, 20 bp, Camellia sinensis
  source 1..20: mol_type = other DNA
acacttacagtctcttgcgg

SEQ ID NO: 4 - DNA, 1001 bp, Camellia sinensis
  source 1..1001: mol_type = genomic DNA
gagtcatgggtttcttaaatttctctaaaaaatatttaggtggtgactctgtatctggcaaaatagtccatttttggcaatttgattcaaaatcagttttccaacatatttgccgaattgggactttttggtgattatctatttcacattgcacatgtgaaatcagattcagaaccgtgggagtccgatactgtagggcttattcgtcttccgaaaaggggcatgcaaagtcgaactacaagtcccctggggaggatggattgcaaaattaccgtacacagtagcaatcccgtctttaaaggcgtactttaccaactgatggaccattgatgacacaaccctcatctgatgtagccagggtcttcccagtagtagattgaaagtgtccgaaacatccatgacatagaatttaacctgatgctcagacgggccgagtaggatatggctcttaaacattaccatgacatcttggctcgtattgtcatataagcctaaacggcntgggtcgtgggcgtaaagttagtcggcctcacaccgatggcataggcggtccttaccgggcatacattaatcgccgatccgttatctaccaacaccactggaatccactttttctgactttccagcgttacatataagggccaattgtggttagcaccctcaggtggtaactctttatctataaaagatatcactggcgtaacatccccggatgtaaccaatgataccaattggtcagcagtggtttcgatagggagtttggtccggttcattgcctctagcagcagtgcctgtctatgctcccgagatgccatgattagcccccagattgatatgtcggcctgaatcttcttaagctgtttcaagaccaggttttcttcaacatccttctcttttgatttctcgacccccactgtccttgatatatgccatcttttagggttatcacccattggtacccctttcggtctagattaccctgactttaaggtctccttctc

SEQ ID NO: 5 - DNA, 20 bp, Camellia sinensis
  source 1..20: mol_type = other DNA
cttcatctccaccacacttc

SEQ ID NO: 6 - DNA, 20 bp, Camellia sinensis
  source 1..20: mol_type = other DNA
aatgtatgcccggtaaggac

SEQ ID NO: 7 - DNA, 1001 bp, Camellia sinensis
  source 1..1001: mol_type = genomic DNA
ataatctttttgtacttgttcaggtggaatgaagcaatcaaccgagagtccaggaacattgaatgctaggtcgtcgatcttccaagtctcctccatccgtgtgattgctgtgcccgctctcagattgtccccaaatcttgagatgatcacacttgattggccagaatgcgcgatcatcgtgccctccaccatccgatagtcctcgattttcgtgcccatggtggtctcccaataggtagggtaggttccgggggactggattctggtgaggtaagagtcctctaaatacactagcagaccacttctttggctgaagtaaccaaacatgacatgcttgatcatctctgcactgttgtcactccgatcggctaggtccgtctgatccgcggacaatttcaacacgaagcaatcgacgctcaagattcgtttttcgcccacgtattgtgctagggaaaacacagccgatacagccacaggatctagtccctgcataaataacantatgttttttacatagaggaaaataatatctgtcacatgaattctactccattttttaaccttctaagaaagtgtggtgaaaaaaatattaaatccattgggtaaaatataacagtctttaacataacaatatggcgaactatacattcaattctagaaaatgtctcatttttatagatttttatgaaagggatcaaccttctttttttttattggaagcactatataaataatgtcaaatagttttccaaacttatctaaataaagttttaataattttaatccacacattttgaatttaatttacttatttttagtagataacattaccacagtcaaaaagagtgccaacatgaacctccagcacacttgaagagcacttgacgatcatattgggaaagttaccagccagcactcccaaaaaaaaaaaaagaaaaaaagataaaagattaaaaaaattagtaaaaagtgactttacaaaaaggaatattccacctctg

SEQ ID NO: 8 - DNA, 20 bp, Camellia sinensis
  source 1..20: mol_type = other DNA
tctctgcactgttgtcactc

SEQ ID NO: 9 - DNA, 20 bp, Camellia sinensis
  source 1..20: mol_type = other DNA
caccacactttcttagaagg

SEQ ID NO: 10 - DNA, 1001 bp, Camellia sinensis
  source 1..1001: mol_type = genomic DNA
aattaataaagacttgaacagtgaggaggacaatggagagagggatttcatggaggagtttcagagaattagcttatttgatgagttgtttattttattttattttatttttacttacagtggtagatgcatcccatccccatcatcgtcccaatcgttattgccatgattttcatgtttcatcaggtgttgcttctcttgtttgtgcttccaactttccatcctctctttccaagctacgctgccatagccataagcagccaaatccttggaaggatccatggatcgagattgcactgcaaaaatgggcaggggattatcatacagatttgaccttcaacgtgggagggaggggagataaaaggaaaccatagcgtagcgtagcatagcataggaaagcaaagcagaattaattaaaattaccgggtaggctaggatctgagaaaggaagtggatgaatccttctgctgctgctgctgctgccaccaccaacacccactntcgatggaaccaatgcatgttgttcaggaggaatatcatcatccacctgcaaacacaaacgctgcaggtctcaggctcctgctgtctgaaatttgcatacaatgatttttagaattccacagcaacagcaacagcaacagcaacggtagtcgtaccatatggccgttggtaaggaggggaagttgaggcaaagtattactattattagtattgtgaaagacatgtgggtgcaattcggatgagtcgaaaatatggccatagctcatgtgcgaaccaccgtgaccgtgaagtatagcctctgaacgagcaagagagtgctgctgtgaatccagtagtttagcactgtcacgaaccctcccttcaaaattgaactcgttttccacatcgtcaatgtcatcttcttcttcatcaccctccactctagcacaccctgcatcattcatccatccattgatcatccgggtagaactaacaaattttaacaaatatcgaatcccccc

SEQ ID NO: 11 - DNA, 20 bp, Camellia sinensis
  source 1..20: mol_type = other DNA
gatttgaccttcaacgtggg

SEQ ID NO: 12 - DNA, 20 bp, Camellia sinensis
  source 1..20: mol_type = other DNA
tgcagcgtttgtgtttgcag

SEQ ID NO: 13 - DNA, 1001 bp, Camellia sinensis
  source 1..1001: mol_type = genomic DNA
cggcgggctgttccaagaaaaaatataaaattaaataagtttgtatattgtcctgccgggaaacaaatgtggaatcattacaaagaattaagagaagcacttacattgctccatcttttatcgagaaattcattgatcgcaatggcgttttgtccgtacatcataggagtcggaagagtgagagagccatctgatgcacactaaagaaggacagaaactgtttgaggaacctgaacattttgaggataagtcaaaaaaagttaattaggtttcggagtccagtgattgtcgaaccaacaaaacaaaacttatatgctgtaaaagaacttcaacttacctagtaatagacggtgcaaacccaattgtatagtaggtaagtacgatccatatcacagattccatgaatgaaacgggaattcggaggagccaaattggcaagctaaaagcccatgcagggaaaaacaagctatccctctgtttaaagaacacgggaagcttacnaaccgtcattgcaagctctgccatcccattgaacattatattaacaagactgaaaaacagcgctcccaaatactttgaagcatcttctactgttccggttttcatttctgttcttaaaaaaacagtgagggcaattgtggccatgattgttatctgagtggttttgaatatgtatgtgaaagagttgcgcttcattagcagccactccctcgataagcatgccttgaagagttcccgattggagatgccataactctcagtcaccaacgcagcagggtgggctttggactggtcataaggaattctaagttcttcagtcatctgttgcccgatgtggaaagagttgaaggcctgtgcaaagtcgttcaccgagacatatctgtaaggttggttctttttgaaccaatactgttcttggtccttcttggaagttacttcttggagaaaatctgcaactcctttccttttggggcatttgaatcccatatattcaaagaact

SEQ ID NO: 14 - DNA, 20 bp, Camellia sinensis
  source 1..20: mol_type = other DNA
gtaatagacggtgcaaaccc

SEQ ID NO: 15 - DNA, 20 bp, Camellia sinensis
  source 1..20: mol_type = other DNA
caaagtatttgggagcgctg

SEQ ID NO: 16 - DNA, 1001 bp, Camellia sinensis
  source 1..1001: mol_type = genomic DNA
ccctacactttttttttaaatggtgagttgtccccacacttcaatatcgcacataatacacgttttcatttcatgtcgtcttcaatacagaagactcgcaccactattagctagcctattatagcccctcctcttaactacctctacccccaattcctctctctctctctctctctctctctctctctctctctctataaaatcaaaaataaggacttgtttgtttcatcgtactttgttttataggatcaaccttggaagccacacctaggcatgagttgctcaatagattggccagaaccaattgtccgtgtccaatccttgtccgacagcggcacccccaccatccccgactgctacgtcaaaccgccacaggaccggccggtagtcaactcctcctccaaccaccatgacaccgatgtaaacatccccttaattgacctcggagttttaacatccggggacgacaatactactctaagagcaaccaccatagcccanatatccgaagcgtgtcgtgagtggggcttcttccaggtggtcaatcacggagtgagcccccacttgatggatcgcgccagggatatctggcgcgatttcttccatcttccaatggaagaaaagcaagtttatgcgaattcacccaaaacgtacgaagggtatggaagtcggttaggcgtccagaaaggtgccattctcgactggagcgactactacttcttgcactttcttccgtgctcgcttaaagatcataacaagtggcccgccttgccagctcctctcaggtgaattgctttaatttttaattttttaatgtaataataatatataaatgttggtgacttgtatactttaatgtaacaaccaccatctatttggactttactgatctaattttatgtattactatattactggttgtgtttagggaagtgatagatgagtacgcggaccacttagtaaagctaagtgggcgattaatgaaggttttgtcaataaat

SEQ ID NO: 17 - DNA, 20 bp, Camellia sinensis
  source 1..20: mol_type = other DNA
ttgtccgtgtccaatccttg

SEQ ID NO: 18 - DNA, 20 bp, Camellia sinensis
  source 1..20: mol_type = other DNA
attgaccacctggaagaagc

SEQ ID NO: 19 - DNA, 1001 bp, Camellia sinensis
  source 1..1001: mol_type = genomic DNA
aatcattaagagtcattatggtaatcatgagcttaattactccaagtaaagccaatcttcatcatagaaataaaaattacaaaaaaaaaaaaaaaaaaaaagtctttcagctgaacaacccatccctgcaactgcaccaccataattgagatctaaatctgaaggaacttgcttgagatctaaatctgaaggaacttgcttgcttaggaacatccacatccatgatttctacaatttttggaagacacagaaccagagaagatgactcaaaatcaagcagcaattgtaagaaaattcgaccaatcgaaatcatcttggaattaatcattgtagcctccttcatctccaccacacttctcctcctacttccatgcgattacgtcgacggcagccctattcccaccatcatattcaaaggactcccctccaccttccacgccttcgtcgtctccctcatcttcgccttctccggagccttgagcgccttgttgatccacgacncatccctctttgccaagctctgcgagttctcttccatggcctccatgacctctgctctctctttgctactttgggctatgttcttcacctgttttcaaccacaacccaggtaaaactcgaattcagacatcacatggtaagaaaacaagttattaaggtttttaaccttataaagactttttttcttttttcttttccttcctgtccaacggacacgtggtgtgttttaaaattaataaatcgtgtatcagatatggatatacaatcgcgtggtcagttgaaattactattggtatgctttatataccgtgtcgtgtgtaaaattaaaacttgttttgtgatgttgttggtctgttatgtacttggtgttgttgaaataatattaccataaatttgaataagcctttattatgtggagatccgatggattaatgatgcatattgtcacagaattcaaaatgatttcattttgagcatggtgacgagggttccaagccctg

SEQ ID NO: 20 - DNA, 20 bp, Camellia sinensis
  source 1..20: mol_type = other DNA
cttcatctccaccacacttc

SEQ ID NO: 21 - DNA, 20 bp, Camellia sinensis
  source 1..20: mol_type = other DNA
gcccaaagtagcaaagagag

SEQ ID NO: 22 - DNA, 1001 bp, Camellia sinensis
  source 1..1001: mol_type = genomic DNA
agggagacttttatcttgagagctagaagaagagaaagttagagaaaagaaagagaagtaggaagaaaatcaaagggaattcacattcgtccttttggagttgagaattgaacacttaggtgatttcgaaaatcataaatgaggtgtgttaaactaatatcgttcagctacagttactcagtaaattctctttctcagaggctacgcaggtgtagtttgagttaaacttggccacttaaactaatggaaccattaggggcccaagctaattagttcctagaacaaaggagagaggacggagaagcatagagaaagttagagagaaacttttttcttgagagatagaagagatagttagagaaaagaaagagaaacgggaaaaaaatcattgggaattcgcattcgtccttttgggcttgagaattgaacagttggggaatttgggaaaccttaaatgcggtgcttatgtttaactaatatcgttaagtgccaattactcantaaatcctctttcttagatgctaagcaagatttagtgtagttaaacttggccacttaagctaatggaacagttagggtcccaagcgaattagtttcctagaacaaaagatagaaggatggagaatgtagcacgttcgtgagggaccccgctactacagttcggactcgatttgtgtcacggttcttaatctgaaccaaagagtccaaatccggcaaatcgttttgagaaacagattttttgaaaagaagtgccaaacatggactgctttgctagatatagagtcgccacctaaatatttttttaaaatggggaaatttaggaaaccctaacttggtgccaaaggccacgtgtccgtcattgccaaagttgcctgggctcgggagcttgggtacgattggggaaggtcagctatgagcaccccctctcgcccgatccgaagatcggcctctactaaccgtgatatccgtttttgaaaacgttatgtgttcttaaaccaatt

SEQ ID NO: 23 - DNA, 20 bp, Camellia sinensis
  source 1..20: mol_type = other DNA
ttcgcattcgtccttttggg

SEQ ID NO: 24 - DNA, 20 bp, Camellia sinensis
  source 1..20: mol_type = other DNA
acgtgctacattctccatcc

(19) United States
(12) Patent Application Publication
(10) Pub. No.: US 2024/0309433 A1
(43) Pub. Date: Sep. 19, 2024

(54) METHOD FOR EVALUATING TEA PLANT (+)-CATECHIN CONTENT

(71) Applicant: TEA RESEARCH INSTITUTE, GUANGDONG ACADEMY OF AGRICULTURAL SCIENCES, Guangdong (CN)

(72) Inventors: Kaixing FANG, Guangdong (CN); Hualing WU, Guangdong (CN); Xiaohui JIANG, Guangdong (CN); Hongjian LI, Guangdong (CN); Qiushuang WANG, Guangdong (CN); Dandan QIN, Guangdong (CN); Chendong PAN, Guangdong (CN); Bo LI, Guangdong (CN)

(73) Assignee: TEA RESEARCH INSTITUTE, GUANGDONG ACADEMY OF AGRICULTURAL SCIENCES, Guangdong (CN)

(21) Appl. No.: 18/662,886

(22) Filed: May 13, 2024

Related U.S. Application Data
(62) Division of application No. 17/254,302, filed on Dec. 21, 2020.

(30) Foreign Application Priority Data
Sep. 4, 2019 (CN) 201910833662.X
Sep. 4, 2019 (CN) 201910833670.4
Sep. 4, 2019 (CN) 201910833687.X
Sep. 4, 2019 (CN) 201910833698.8
Sep. 4, 2019 (CN) 201910834177.4

(51) Int. Cl. C12Q 1/6827

(52) U.S. Cl. CPC C12Q 1/6827; C12Q 2600/156

(57) ABSTRACT

A molecular marker combination linked to quantitative traits of tea plant (+)-catechin content, including a SNP site 1, a SNP site 2, a SNP site 3, a SNP site 4, a SNP site 5, a SNP site 6, a SNP site 7 and a SNP site 8, which are located in tea genomes Scaffold4239:309117, Scaffold3614: 66549, Scaffold349: 3413816, Scaffold1989: 2316385, Scaffold451: 940283, Scaffold3727:442660, Scaffold115:803980 and Scaffold920:281727, respectively, and genotypes thereof are extremely significantly correlated with the (+)-catechin content is provided. A detection method for detecting each site, and one or more molecular marker site is used to evaluate the tea plant (+)-catechin content.

### CROSS-REFERENCE TO RELATED APPLICATION

This application is a divisional application of and claims the priority benefit of U.S. application Ser. No. 17/254,302, filed on Dec. 21, 2020, now pending. The prior U.S. application Ser. No. 17/254,302 is a 371 of international application of PCT application serial no. PCT/CN2019/110920, filed on Oct. 14, 2019, which claims the priority benefit of China application no. 201910833687.X, China application no. 201910834177.4, China application no. 201910833698.8, China application no. 201910833662.X, and China application no. 201910833670.4, filed on Sep. 4, 2019. The entirety of each of the above mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Mar. 27, 2024, is named 102274-us-sequence_listing and is 29,861 bytes in size.

### TECHNICAL FIELD

The present invention relates to the technical field of molecular genetics and breeding, and more specifically, to a molecular marker combination linked to quantitative traits of tea plant (+)-catechin content.

### BACKGROUND

Tea (Camellia sinensis (L.) O. Kuntze) belongs to the genus Camellia (Theaceae), which originated in southwest China, with a cultivation history of more than 5,000 years. Tea, coffee, and cocoa are collectively referred to as the world's three major non-alcoholic beverages, which have important economic value and have an important impact on society and culture.

(+)-Catechin (C) is an important secondary metabolite in tea plant that affects flavor. It not only affects tea quality, but also has a variety of physiological functions. Studies have shown that (+)-catechin is an important health component of tea and has multiple functions such as preventing and treating cardiovascular disease and preventing cancer. It is a reducing polyphenolic substance that is easily oxidized by air in aqueous solutions and is often used as an antioxidant. Studies have shown that (+)-catechin (C) can inhibit the proliferation and migration of human liver cancer cells (HepG2) and induce apoptosis of the of human liver cancer cells. Dextro-catechin ((+)-catechin) also has various effects such as reducing capillary permeability, anti-diarrhea, hemostatic, anti-virus, fungicidal, inhibiting angiotensin converting enzyme (ACE) and preventing gastric ulcers. (+)-Catechin (C) has protective effects on dyslipidemia caused by iron overload. (+)-Catechin (C) can improve learning and memory disorder in mice caused by aluminum overload, and has strong antioxidant capacity.

Based on the importance of (+)-catechin to tea quality and physiological functions, it is of great significance to breed tea plant resources with specific (+)-catechin content. At present, tea plant breeding is mainly carried out by conventional methods, and excellent individual plants are selected from wild populations and hybrid offspring for systematic breeding. This method is time-consuming and inefficient, which makes the replacement of new varieties slow, and it cannot quickly meet the public's demand for new products. Since molecular marker-assisted breeding can select breeding materials at the seedling stage, it can significantly improve breeding efficiency.

The discovery of molecular markers closely linked to the excellent traits of the tea plant is the basis for the development of molecular marker-assisted selection breeding for the tea plant. However, due to the limitation of the research progress of traditional quantitative trait locus (QTL) mapping, it has not been able to find a SNP molecular marker site that affects the (+)-catechin content.

### SUMMARY OF THE INVENTION

Objectives of the present invention are to overcome the shortcomings of the prior art and provide a molecular marker combination linked to quantitative traits of tea plant (+)-catechin content.

The first objective of the present invention is to provide a molecular marker combination linked to quantitative traits of tea plant (+)-catechin content. The molecular marker combination comprises a SNP site 1, a SNP site 2, a SNP site 3, a SNP site 4, a SNP site 5, a SNP site 6, a SNP site 7 and a SNP site 8, which are located in tea genomes Scaffold4239:309117, Scaffold3614: 66549, Scaffold349: 3413816, Scaffold1989: 2316385, Scaffold451: 940283, Scaffold3727:442660, Scaffold115:803980 and Scaffold920:281727, respectively, which are a 501st base of a nucleotide sequence shown in SEQ ID NO: 1, a 501 st base of a nucleotide sequence shown in SEQ ID NO:4, a 501st base of a nucleotide sequence shown in SEQ ID NO:7, a 501st base of a nucleotide sequence shown in SEQ ID NO: 10, a 501st base of a nucleotide sequence shown in SEQ ID NO: 13, a 501st base of a nucleotide sequence shown in SEQ ID NO: 16, a 501st base of a nucleotide sequence shown in SEQ ID NO: 19, and a 501st base of a nucleotide sequence shown in SEQ ID NO:22.

The second objective of the present invention is to provide use of any one or more molecular marker of the molecular marker combination in evaluating the tea plant (+)-catechin content.

The third objective of the present invention is to provide use of primers of any one or more molecular marker of the molecular marker combination in evaluating the tea plant (+)-catechin content.

The fourth objective of the present invention is to provide primers for detecting SNP site 1.

The fifth objective of the present invention is to provide primers for detecting SNP site 2.

The sixth objective of the present invention is to provide primers for detecting SNP site 3.

The seventh objective of the present invention is to provide primers for detecting SNP site 4.

The eighth objective of the present invention is to provide primers for detecting SNP site 5.

The ninth objective of the present invention is to provide primers for detecting SNP site 6.

The tenth objective of the present invention is to provide primers for detecting SNP site 7.

The eleventh objective of the present invention is to provide primers for detecting SNP site 8.

The twelfth objective of the present invention is to provide a kit for evaluating tea plant (+)-catechin content.

The thirteenth objective of the present invention is to provide a method for evaluating tea plant (+)-catechin content.

The fourteenth objective of the present invention is to provide use of any one or more of any one or more molecular marker in the molecular marker combination, the primers for the SNP site 1, the primers for the SNP site 2, the primers for the SNP site 3, the primers for the SNP site 4, the primers for the SNP site 5, the primers for the SNP site 6, the primers for the SNP site 7, the primers for the SNP site 8, or the kit in molecular-assisted breeding.

In order to achieve the above objectives, the present invention is realized by the following technical solutions.

After a long period of exploratory research, the inventors discovered eight SNP site molecular markers linked to (+)-catechin content. It is further used to establish a detection method for detecting the sites, which can be used to evaluate the tea plant (+)-catechin content, for further use in resource screening and molecular breeding.

Therefore, the present invention claims a molecular marker combination linked to quantitative traits of tea plant (+)-catechin content, including a SNP site 1, a SNP site 2, a SNP site 3, a SNP site 4, a SNP site 5, a SNP site 6, a SNP site 7 and a SNP site 8, which are located in tea genomes Scaffold4239:309117, Scaffold3614: 66549, Scaffold349: 3413816, Scaffold1989: 2316385, Scaffold451: 940283, Scaffold3 727:442660, Scaffold115:803980 and Scaffold920:281727, respectively, i.e., a 501st base of a nucleotide sequence shown in SEQ ID NO:1, a 501st base of a nucleotide sequence shown in SEQ ID NO:4, a 501st base of a nucleotide sequence shown in SEQ ID NO:7, a 501 st base of a nucleotide sequence shown in SEQ ID NO:10, a 501st base of a nucleotide sequence shown in SEQ ID NO:13, a 501st base of a nucleotide sequence shown in SEQ ID NO: 16, a 501st base of a nucleotide sequence shown in SEQ ID NO: 19, and a 501st base of a nucleotide sequence shown in SEQ ID NO:22.

The SNP site 1 is located in the tea genome Scaffold4239:309117 (i.e. the 501st base of the nucleotide sequence shown in SEQ ID NO:1), this site is G or A, and genotype thereof is extremely significantly correlated with the (+)-catechin content in the dry matter of the tea plant. It is shown by correlation analysis and significance analysis verification that the (+)-catechin content in the dry matter of tea soup corresponding to an AA genotype sample has extremely significant difference compared with GG and GA genotype samples. It is statistically judged that, when the genotype of the sample is double mutant AA, the catechin content in the dry matter in the tea plant is more likely to be higher than the normal average of the sample of which the genotype is wild type GG or single mutant GA.

The SNP site 2 is located in the tea genome Scaffold3614: 66549 (i.e. the 501st base of the nucleotide sequence shown in SEQ ID NO:4), this site is T or C, and genotype thereof is extremely significantly correlated with the (+)-catechin content in the dry matter of the tea plant. It is shown by correlation analysis and significance analysis verification that the (+)-catechin content in the dry matter corresponding to a CC genotype sample has extremely significant difference compared with TT and CT genotype samples. It is statistically judged that, when the genotype of the sample is double mutant CC, the (+)-catechin content in the dry matter in the tea plant is more likely to be higher than the sample of which the genotype is wild type TT or single mutant CT.

The SNP site 3 is located in the tea genome Scaffold349: 3413816 (i.e. the 501st base of the nucleotide sequence shown in SEQ ID NO:7), this site is G or A, and genotype thereof is extremely significantly correlated with the (+)-catechin content in the dry matter of the tea plant. It is shown by correlation analysis and significance analysis verification that the (+)-catechin content in the dry matter of tea soup corresponding to a GG genotype sample has extremely significant difference compared with GA and AA genotype samples. It is statistically judged that, when the genotype of the sample is double mutant GG, the (+)-catechin content in the dry matter in the tea plant is more likely to be higher than the sample of which the genotype is wild type AA or single mutant GA.

The SNP site 4 is located in the tea genome Scaffold1989: 2316385 (i.e. the 501st base of the nucleotide sequence shown in SEQ ID NO: 10), this site is G or A, and genotype thereof is extremely significantly correlated with the (+)-catechin content in the dry matter of the tea plant. It is shown by correlation analysis and significance analysis verification that the (+)-catechin content in the dry matter of tea soup corresponding to an AA genotype sample has extremely significant difference compared with GA and GG genotype samples. It is statistically judged that, when the genotype of the sample is double mutant AA, the (+)-catechin content in the dry matter in the tea plant is more likely to be higher than the normal average of the sample of which the genotype is wild type GG or single mutant GA.

The SNP site 5 is located in the tea genome Scaffold451: 940283 (i.e. the 501st base of the nucleotide sequence shown in SEQ ID NO:13), this site is C or T, and genotype thereof is extremely significantly correlated with the (+)-catechin content in the dry matter of the tea plant. It is shown by correlation analysis and significance analysis verification that the (+)-catechin content in the dry matter of tea soup corresponding to a TT genotype sample has extremely significant difference compared with CC and CT genotype samples. It is statistically judged that, when the genotype of the sample is double mutant TT, the (+)-catechin content in the dry matter in the tea plant is more likely to be higher than the normal average of the sample of which the genotype is wild type CC or single mutant CT.

The SNP site 6 is located in the tea genome Scaffold3727:442660 (i.e. the 501st base of the nucleotide sequence shown in SEQ ID NO: 16), this site is G or A, and genotype thereof is extremely significantly correlated with the (+)-catechin content in the dry matter of the tea plant. It is shown by correlation analysis and significance analysis verification that the (+)-catechin content in the dry matter of tea soup corresponding to an AA genotype sample has extremely significant difference compared with GG and GA genotype samples. It is statistically judged that, when the genotype of the sample is double mutant AA, the (+)-catechin content in the dry matter in the tea plant is more likely to be higher than the normal average of the sample of which the genotype is wild type GG or single mutant GA.

The SNP site 7 is located in the tea genome Scaffold115: 803980 (i.e. the 501st base of the nucleotide sequence shown in SEQ ID NO:19), this site is G or A, and genotype thereof is extremely significantly correlated with the (+)-catechin content in the dry matter of the tea plant. It is shown by correlation analysis and significance analysis verification that the (+)-catechin content in the dry matter of the tea plant corresponding to a GG genotype sample has extremely significant difference compared with AA and GA genotype samples, it is statistically judged that, when the genotype of the sample is double mutant GG, the (+)-catechin content in the dry matter in the tea plant is more likely to be higher than the sample of which the genotype is wild type AA or single mutant GA.

The SNP site 8 is located in the tea genome Scaffold920: 281727 (i.e. the 501st base of the nucleotide sequence shown in SEQ ID NO:22), this site is G or A, and genotype thereof is extremely significantly correlated with the (+)-catechin content in the dry matter of the tea plant. It is shown by correlation analysis and significance analysis verification that the (+)-catechin content in the dry matter of the tea plant corresponding to a GG genotype sample has extremely significant difference compared with AA and GA genotype samples, it is statistically judged that, when the genotype of the sample is double mutant GG, the (+)-catechin content in the dry matter in the tea plant is more likely to be higher than the sample of which the genotype is wild type AA or single mutant GA.

The tea plant (+)-catechin content according to the present invention is specifically a proportion of (+)-catechin in dry matter of fresh tea leaves.

Use of any one or more molecular marker of the molecular marker combination in evaluating the tea plant (+)-catechin content also belongs to the scope of protection of the present invention.

The present invention further claims use of primers of any one or more molecular marker of the molecular marker combination in evaluating the tea plant (+)-catechin content.

Primers for the SNP site 1, wherein nucleotide sequences thereof are shown as SEQ ID NO: 2 and SEQ ID NO: 3.

| (SEQ ID NO: 2) | |
|---|---|
| primer F: | GAAGACTAACCCGTATCGAG; |
| (SEQ ID NO: 3) | |
| primer R: | ACACTTACAGTCTCTTGCGG. |

Primers for the SNP site 2, wherein nucleotide sequences thereof are shown as SEQ ID NO: 5 and SEQ ID NO: 6.

| (SEQ ID NO: 5) | |
|---|---|
| primer F: | GATGACACAACCCTCATCTG; |
| (SEQ ID NO: 6) | |
| primer R: | AATGTATGCCCGGTAAGGAC. |

Primers for the SNP site 3, wherein nucleotide sequences thereof are shown as SEQ ID NO: 8 and SEQ ID NO: 9.

| (SEQ ID NO: 8) | |
|---|---|
| primer F: | TCTCTGCACTGTTGTCACTC; |
| (SEQ ID NO: 9) | |
| primer R: | CACCACACTTTCTTAGAAGG. |

Primers for the SNP site 4, wherein nucleotide sequences thereof are shown as SEQ ID NO: 11 and SEQ ID NO: 12.

| (SEQ ID NO: 11) | |
|---|---|
| primer F: | GATTTGACCTTCAACGTGGG; |
| (SEQ ID NO: 12) | |
| primer R: | TGCAGCGTTTGTGTTTGCAG. |

Primers for the SNP site 5, wherein nucleotide sequences thereof are shown as SEQ ID NO: 14 and SEQ ID NO: 15.

| (SEQ ID NO: 14) | |
|---|---|
| primer F: | GTAATAGACGGTGCAAACCC; |
| (SEQ ID NO: 15) | |
| primer R: | CAAAGTATTTGGGAGCGCTG. |

Primers for the SNP site 6, wherein nucleotide sequences thereof are shown as SEQ ID NO: 17 and SEQ ID NO: 18.

| (SEQ ID NO: 17) | |
|---|---|
| primer F: | TTGTCCGTGTCCAATCCTTG; |
| (SEQ ID NO: 18) | |
| primer R: | ATTGACCACCTGGAAGAAGC. |

Primers for the SNP site 7, wherein nucleotide sequences thereof are shown as SEQ ID NO: 20 and SEQ ID NO: 21.

| (SEQ ID NO: 20) | |
|---|---|
| primer F: | CTTCATCTCCACCACACTTC; |
| (SEQ ID NO: 21) | |
| primer R: | GCCCAAAGTAGCAAAGAGAG. |

Primers for the SNP site 8, wherein nucleotide sequences thereof are shown as SEQ ID NO: 23 and SEQ ID NO: 24.

| (SEQ ID NO: 23) | |
|---|---|
| primer F: | TTCGCATTCGTCCTTTTGGG; |
| (SEQ ID NO: 24) | |
| primer R: | ACGTGCTACATTCTCCATCC. |

Further, the present invention claims a kit for evaluating tea plant (+)-catechin content, including a reagent for detecting the molecular marker combination or any one molecular marker thereof.

Preferably, the reagent is the primers for the SNP site 1 which have the nucleotide sequences shown as SEQ ID NO: 2 and SEQ ID NO: 3, the primers for the SNP site 2 which have the nucleotide sequences shown as SEQ ID NO: 5 and SEQ ID NO: 6, the primers for the SNP site 3 which have the nucleotide sequences shown as SEQ ID NO: 8 and SEQ ID NO: 9, the primers for the SNP site 4 which have the nucleotide sequences shown as SEQ ID NO: 11 and SEQ ID NO: 12, the primers for the SNP site 5 which have the nucleotide sequences shown as SEQ ID NO: 14 and SEQ ID NO: 15, the primers for the SNP site 6 which have the nucleotide sequences shown as SEQ ID NO: 17 and SEQ ID NO: 18, the primers for the SNP site 7 which have the nucleotide sequences shown as SEQ ID NO: 20 and SEQ ID NO: 21, and/or the primers for the SNP site 8 which have the nucleotide sequences shown as SEQ ID NO: 23 and SEQ ID NO: 24.

The most preferably, the kit contains the primers for the SNP site 1 have the nucleotide sequences shown as SEQ ID NO: 2 and SEQ ID NO: 3, the primers for the SNP site 2 have the nucleotide sequences shown as SEQ ID NO: 5 and SEQ ID NO: 6, the primers for the SNP site 3 have the nucleotide sequences shown as SEQ ID NO: 8 and SEQ ID NO: 9, the primers for the SNP site 4 have the nucleotide sequences shown as SEQ ID NO: 11 and SEQ ID NO: 12, the primers for the SNP site 5 have the nucleotide sequences shown as SEQ ID NO: 14 and SEQ ID NO: 15, and/or the primers for the SNP site 6 have the nucleotide sequences shown as SEQ ID NO: 17 and SEQ ID NO: 18, the primers for the SNP site 7 have the nucleotide sequences shown as SEQ ID NO: 20 and SEQ ID NO: 21, the primers for the SNP site 8 have the nucleotide sequences shown as SEQ ID NO: 23 and SEQ ID NO: 24, 2×Taq PCR Master Mix, and ddH₂O.

A usage method is as follows:

(1) CTAB method is used to extract total DNA from buds of tea plant, it is ensured that A260/A280 of each DNA sample is between 1.8 and 2.0, and the concentration is greater than 100 μg/μl;

(2) PCR amplification

PCR system (10 μl) is as follows:

| | |
|---|---|
| 2× Taq PCR | 5 μl |
| Master Mix primers | Each 0.5 μl |
| DNA template | 1 μl |
| ddH₂O | 3 μl |

PCR amplification procedure is as follows:

| | | |
|---|---|---|
| 95° C. | 5 minutes | |
| 95° C. | 30 seconds | ×45 cycles |
| 56° C. | 30 seconds | |
| 72° C. | 30 seconds | |
| 72° C. | 2 minutes | |
| 4° C. | forever | |

(3) Product purification

The PCR amplification products are subjected to gel electrophoresis, followed by recovery and purification using a commercially available gel electrophoresis DNA recovery kit.

A band with a fragment length of about 240 bp in the amplification product of the primers shown in SEQ ID NO: 2 and SEQ ID NO: 3 is selected for recovery and purification.

A band with a fragment length of about 240 bp in the amplification product of the primers shown in SEQ ID NO: 5 and SEQ ID NO: 6 is selected for recovery and purification.

A band with a fragment length of about 240 bp in the amplification product of the primers shown in SEQ ID NO: 8 and SEQ ID NO: 9 is selected for recovery and purification.

A band with a fragment length of about 240 bp in the amplification product of the primers shown in SEQ ID NO: 11 and SEQ ID NO: 12 is selected for recovery and purification.

A band with a fragment length of about 240 bp in the amplification product of the primers shown in SEQ ID NO: 14 and SEQ ID NO: 15 is selected for recovery and purification.

A band with a fragment length of about 240 bp in the amplification product of the primers shown in SEQ ID NO: 17 and SEQ ID NO: 18 is selected for recovery and purification.

A band with a fragment length of about 240 bp in the amplification product of the primers shown in SEQ ID NO: 20 and SEQ ID NO: 21 is selected for recovery and purification.

A band with a fragment length of about 240 bp in the amplification product of the primers shown in SEQ ID NO: 23 and SEQ ID NO: 24 is selected for recovery and purification.

(4) Sequencing and interpretation of results

The recovered and purified product is sent to a sequencing company for Sanger sequencing. At the site Scaffold4239:309117, it is statistically judged that, when the genotype of the sample is double mutant AA, the (+)-catechin content in the dry matter in the tea plant is more likely to be higher than the normal average of the sample of which the genotype is wild type GG or single mutant GA.

At the site Scaffold3614: 66549, when the genotype is double mutant CC, the (+)-catechin content in the tea plant is more likely to be higher than the normal average of CT and TT genotype resources.

At the site Scaffold349: 3413816, when the genotype is double mutant GG, the (+)-catechin content in the tea plant is more likely to be higher than the normal average of AA and GA genotype resources.

At the site Scaffold1989: 2316385, when the genotype is double mutant AA, the (+)-catechin content in the tea plant is more likely to be higher than the normal average of GG and GA genotype resources.

At the site Scaffold451: 940283, it is statistically judged that, when the genotype of the sample is double mutant TT, the (+)-catechin content in the dry matter in the tea plant is more likely to be higher than the normal average of the sample of which the genotype is wild type CC or single mutant CT.

At the site Scaffold3727:442660, it is statistically judged that, when the genotype of the sample is double mutant AA, the (+)-catechin content in the dry matter in the tea plant is more likely to be higher than the normal average of the sample of which the genotype is wild type GG or single mutant GA.

At the site Scaffold115: 803980, it is statistically judged that, when the genotype of the sample is double mutant GG, the (+)-catechin content in the dry matter in the tea plant is more likely to be higher than the sample of which the genotype is wild type AA or single mutant GA.

At the site Scaffold920: 281727, it is statistically judged that, when the genotype of the sample is double mutant GG, the (+)-catechin content in the dry matter in the tea plant is more likely to be higher than the sample of which the genotype is wild type AA or single mutant GA.

In the meantime, the present invention claims a method for evaluating the tea plant (+)-catechin content, which detects a genotype of any one or more molecular marker of the molecular marker combination.

Use of any one or more of any one or more molecular markers of the molecular marker combination, the primers for the SNP site 1, the primers for the SNP site 2, the primers for the SNP site 3, the primers for the SNP site 4, the primers for the SNP site 5, the primers for the SNP site 6, the primers for the SNP site 7, the primers for the SNP site 8, or the kit in molecular-assisted breeding.

Compared with the prior art, the present invention has the following beneficial effects.

The present invention first discovered the following.

The SNP site 1 is located in the tea genome Scaffold4239:309117, this site is G or A, and genotype thereof is extremely significantly correlated with the (+)-catechin content in the dry matter of the tea plant. It is shown by correlation analysis and significance analysis verification that the (+)-catechin content in the dry matter of tea soup corresponding to an AA genotype sample has extremely significant difference compared with GG and GA genotype samples. It is statistically judged that, when the genotype of the sample is double mutant AA, the catechin content in the dry matter in the tea plant is more likely to be higher than the normal average of the sample of which the genotype is wild type GG or single mutant GA.

SNP site 2 is located in the tea genome Scaffold3614: 66549, this site is T or C, and genotype thereof is extremely significantly correlated with the (+)-catechin content in the dry matter of the tea plant. It is shown by correlation analysis and significance analysis verification that the (+)-catechin content in the dry matter corresponding to a CC genotype sample has extremely significant difference compared with TT and CT genotype samples. It is statistically judged that, when the genotype of the sample is double mutant CC, the (+)-catechin content in the dry matter in the tea plant is more likely to be higher than the sample of which the genotype is wild type TT or single mutant CT.

SNP site 3 is located in the tea genome Scaffold349: 3413816, this site is G or A, and genotype thereof is extremely significantly correlated with the (+)-catechin content in the dry matter of the tea plant. It is shown by correlation analysis and significance analysis verification that the (+)-catechin content in the dry matter of tea soup corresponding to a GG genotype sample has extremely significant difference compared with GA and AA genotype samples. It is statistically judged that, when the genotype of the sample is double mutant GG, the (+)-catechin content in the dry matter in the tea plant is more likely to be higher than the sample of which the genotype is wild type AA or single mutant GA.

SNP site 4 is located in the tea genome Scaffold1989: 2316385, this site is G or A, and genotype thereof is extremely significantly correlated with the (+)-catechin content in the dry matter of the tea plant. It is shown by correlation analysis and significance analysis verification that the (+)-catechin content in the dry matter of tea soup corresponding to an AA genotype sample has extremely significant difference compared with GA and GG genotype samples. It is statistically judged that, when the genotype of the sample is double mutant AA, the (+)-catechin content in the dry matter in the tea plant is more likely to be higher than the normal average of the sample of which the genotype is wild type GG or single mutant GA.

SNP site 5 is located in the tea genome Scaffold451: 940283, this site is C or T, and genotype thereof is extremely significantly correlated with the (+)-catechin content in the dry matter of the tea plant. It is shown by correlation analysis and significance analysis verification that the (+)-catechin content in the dry matter of tea soup corresponding to a TT genotype sample has extremely significant difference compared with CC and CT genotype samples. It is statistically judged that, when the genotype of the sample is double mutant TT, the (+)-catechin content in the dry matter in the tea plant is more likely to be higher than the normal average of the sample of which the genotype is wild type CC or single mutant CT.

SNP site 6 is located in the tea genome Scaffold3727:442660, this site is G or A, and genotype thereof is extremely significantly correlated with the (+)-catechin content in the dry matter of the tea plant. It is shown by correlation analysis and significance analysis verification that the (+)-catechin content in the dry matter of tea soup corresponding to an AA genotype sample has extremely significant difference compared with GG and GA genotype samples. It is statistically judged that, when the genotype of the sample is double mutant AA, the (+)-catechin content in the dry matter in the tea plant is more likely to be higher than the normal average of the sample of which the genotype is wild type GG or single mutant GA.

SNP site 7 is located in the tea genome Scaffold 115: 803980, this site is G or A, and genotype thereof is extremely significantly correlated with the (+)-catechin content in the dry matter of the tea plant. It is shown by correlation analysis and significance analysis verification that the (+)-catechin content in the dry matter of the tea plant corresponding to a GG genotype sample has extremely significant difference compared with AA and GA genotype samples, it is statistically judged that, when the genotype of the sample is double mutant GG, the (+)-catechin content in the dry matter in the tea plant is more likely to be higher than the sample of which the genotype is wild type AA or single mutant GA.

SNP site 8 is located in the tea genome Scaffold920: 281727, this site is G or A, and genotype thereof is extremely significantly correlated with the (+)-catechin content in the dry matter of the tea plant. It is shown by correlation analysis and significance analysis verification that the (+)-catechin content in the dry matter of the tea plant corresponding to a GG genotype sample has extremely significant difference compared with AA and GA genotype samples, it is statistically judged that, when the genotype of the sample is double mutant GG, the (+)-catechin content in the dry matter in the tea plant is more likely to be higher than the sample of which the genotype is wild type AA or single mutant GA.

It is further established a detection method for detecting the eight SNP sites, which can be used to evaluate the (+)-catechin content of the tea plant, for further use in screening of tea plant resources and molecular breeding. This is the basis for molecular marker-assisted selective breeding for tea plant, which has great research value.

### BRIEF DESCRIPTION OF THE DRAWINGS

FIG. 1 shows (+)-catechin content in different seasons.

FIG. 2 shows a schematic diagram of a site Scaffold4239:309117 (as shown in SEQ ID NO:1) and primers (as shown in SEQ ID NO:2 and SEQ ID NO:3), wherein N denotes a base to be tested at Scaffold4239:309117, and bold and underlined parts denote upstream and downstream primers.

FIG. 3 shows a schematic diagram of a site Scaffold3614: 66549 (as shown in SEQ ID NO:4) and primers (as shown in SEQ ID NO:5 and SEQ ID NO:6), wherein N denotes a base to be tested at Scaffold3614: 66549, and bold and underlined parts denote upstream and downstream primers.

FIG. 4 shows a schematic diagram of a site Scaffold349: 3413816 (as shown in SEQ ID NO:7) and primers (as shown in SEQ ID NO:8 and SEQ ID NO:9), wherein N denotes a base to be tested at Scaffold349: 3413816, and bold and underlined parts denote upstream and downstream primers.

FIG. 5 shows a schematic diagram of a site Scaffold1989: 2316385 (as shown in SEQ ID NO:10) and primers (as shown in SEQ ID NO:11 and SEQ ID NO:12), wherein N denotes a base to be tested at Scaffold1989: 2316385, and bold and underlined parts denote upstream and downstream primers.

FIG. 6 shows a schematic diagram of a site Scaffold451: 940283 (as shown in SEQ ID NO: 13) and primers (as shown in SEQ ID NO: 14 and SEQ ID NO:15), wherein N denotes a base to be tested at Scaffold451: 940283, and bold and underlined parts denote upstream and downstream primers.

FIG. 7 shows a schematic diagram of a site Scaffold3727:442660 (as shown in SEQ ID NO:16) and primers (as shown in SEQ ID NO:17 and SEQ ID NO:18), wherein N denotes a base to be tested at Scaffold3727:442660, and bold and underlined parts denote upstream and downstream primers.

FIG. 8 shows a schematic diagram of a site Scaffold115: 803980 (as shown in SEQ ID NO:19) and primers (as shown in SEQ ID NO:20 and SEQ ID NO:21), wherein N denotes a base to be tested at Scaffold115: 803980, and bold and underlined parts denote upstream and downstream primers.

FIG. 9 shows a schematic diagram of a site Scaffold920: 281727 (as shown in SEQ ID NO:22) and primers (as shown in SEQ ID NO:23 and SEQ ID NO:24), wherein N denotes a base to be tested at Scaffold920: 281727, and bold and underlined parts denote upstream and downstream primers.

FIG. 10 shows SNAPshot sequencing results of genotype of the sample 2-72 at the site Scaffold4239:309117.

FIG. 11 shows SNAPshot sequencing results of genotype of the sample 2-78 at the site Scaffold4239:309117.

FIG. 12 shows SNAPshot sequencing results of genotype of the sample 2-97 at the site Scaffold4239:309117.

FIG. 13 shows SNAPshot sequencing results of genotype of the sample 2-62 at the site Scaffold1989: 2316385 (reverse compliment).

FIG. 14 shows SNAPshot sequencing results of genotype of the sample 2-77 at the site Scaffold1989: 2316385 (reverse compliment).

FIG. 15 shows SNAPshot sequencing results of genotype of the sample 2-69 at the site Scaffold1989: 2316385 (reverse compliment).

FIG. 16 shows SNAPshot sequencing results of genotype of the sample 2-22 at the site Scaffold3614: 66549 (reverse compliment).

FIG. 17 shows SNAPshot sequencing results of genotype of the sample 2-14 at the site Scaffold3614: 66549 (reverse compliment).

FIG. 18 shows SNAPshot sequencing results of genotype of the sample 2-24 at the site Scaffold3614: 66549 (reverse compliment).

FIG. 19 shows SNAPshot sequencing results of genotype of the sample 2-15 at the site Scaffold349: 3413816.

FIG. 20 shows SNAPshot sequencing results of genotype of the sample 2-19 at the site Scaffold349: 3413816.

FIG. 21 shows SNapshot sequencing results of genotype of the sample 2-66 at the site Scaffold349: 3413816.

FIG. 22 shows SNAPshot sequencing results of genotype of the sample 2-92 at the site Scaffold451: 940283.

FIG. 23 shows SNAPshot sequencing results of genotype of the sample 2-77 at the site Scaffold451: 940283.

FIG. 24 shows SNAPshot sequencing results of genotype of the sample 2-97 at the site Scaffold451: 940283.

FIG. 25 shows SNAPshot sequencing results of genotype of the sample 2-51 at the site Scaffold3727:442660.

FIG. 26 shows SNAPshot sequencing results of genotype of the sample 2-35 at the site Scaffold3727:442660.

FIG. 27 shows SNAPshot sequencing results of genotype of the sample 2-44 at the site Scaffold3727:442660.

FIG. 28 shows SNAPshot sequencing results of genotype of the sample 2-50 at the site Scaffold115: 803980 (reverse compliment).

FIG. 29 shows SNAPshot sequencing results of genotype of the sample 2-97 at the site Scaffold115: 803980 (reverse compliment).

FIG. 30 shows SNAPshot sequencing results of genotype of the sample 2-94 at the site Scaffold115: 803980 (reverse compliment).

FIG. 31 shows SNAPshot sequencing results of genotype of the sample 2-93 at the site Scaffold920: 281727 (reverse compliment).

FIG. 32 shows SNAPshot sequencing results of genotype of the sample 2-94 at the site Scaffold920: 281727 (reverse compliment).

FIG. 33 shows SNAPshot sequencing results of genotype of the sample 2-98 at the site Scaffold920: 281727 (reverse compliment).

FIG. 34 shows sequencing results of genotype at the site Scaffold4239:309117 (the nucleotide sequence shown in SEQ ID NO:1).

FIG. 35 shows sequencing results of genotype at the site Scaffold1989: 2316385 (the nucleotide sequence shown in SEQ ID NO:10).

FIG. 36 shows sequencing results of genotype at the site Scaffold349: 3413816 (the nucleotide sequence shown in SEQ ID NO:7).

FIG. 37 shows sequencing results of genotype at the site Scaffold115: 803980 (the nucleotide sequence shown in SEQ ID NO:19).

FIG. 38 shows sequencing results of genotype at the site Scaffold920: 281727 (the nucleotide sequence shown in SEQ ID NO:22).

### DETAILED DESCRIPTION OF THE INVENTION

The present invention will be further described in detail below with reference to the accompanying drawings and specific embodiments, and the embodiments are only used to explain the present invention, and are not used to limit the scope of the present invention. The test methods used in the following embodiments are all conventional methods unless otherwise specified. The materials and agents used, unless otherwise specified, are the agents and materials available from commercial sources.

### Embodiment 1

### I. Experiment Sample

191 tea plant materials located in Guangdong Province Tea Plant Germplasm Resource Bank (Yingde, Guangdong, 113.30E, 24.3ON) were collected, including 124 from Guangdong, 20 from Fujian, 14 from Guangxi, 9 from Zhejiang, 6 from Hunan, 6 from Yunnan, 1 from Jiangxi, 1 from Guizhou, 1 from Taiwan, and 8 offspring of Kenyan tea, 1 offspring of Georgian species. The selected materials are widely representative.

The selected resources are randomly distributed in the resource bank. Double row per plant was used, each row is 4 m, the row spacing is 1.5 m, and the plant spacing is 35 cm. The resource bank was subjected to conventional water and fertilizer management. At the end of 2016, the resources were pruned and deep pits were applied with base fertilizer, 4 tons of organic fertilizer, 0.75 tons of peanut bran and 5 kg of compound fertilizer per acre. After picking spring tea and summer tea in 2017, pruning and topdressing outside the root were conducted, 15 kg compound fertilizer and 30 kg urea per acre. On Mar. 15, 2017, Jun. 25, 2017, and Sep. 28, 2017, the new shoots (one bud with two leaves) of the tea plant were picked, to make steamed green samples, and tea soup was prepared according to water extraction method.

### II. Phenotypic Data Analysis

### 1. Experimental Procedure

The high-performance liquid chromatography was used to detect (+)-catechin in tea soup related to the taste of tea plant, referring to the Chinese standard detection method.

### 2. Experimental Results

(+)-Catechin content is shown in Table 1.

TABLE 1
Percentage of CAF in dry matter from different
tea plant resources in different seasons

| | (+)-Catechin content (%) | | |
|---|---|---|---|
| Sample | Spring | Summer | Autumn |
| Sample 1 | 1.05 | 1.09 | 1.22 |
| Sample 2 | 1.17 | 1.06 | 1.13 |
| Sample 3 | 1.10 | 1.43 | 1.45 |
| Sample 4 | 1.01 | 1.24 | 1.07 |
| Sample 5 | 0.93 | 1.00 | 0.99 |
| Sample 6 | 1.19 | 1.59 | 1.34 |
| Sample 7 | 1.02 | 1.26 | 1.29 |
| Sample 8 | 1.01 | 1.24 | 1.33 |
| Sample 9 | 1.01 | 1.08 | 1.15 |
| Sample 10 | 0.96 | 1.07 | 1.16 |
| Sample 11 | 1.21 | 1.46 | 1.51 |
| Sample 12 | 0.99 | 1.18 | 1.08 |
| Sample 13 | 0.95 | 1.09 | 1.14 |
| Sample 14 | 0.89 | 1.21 | 1.39 |
| Sample 15 | 1.10 | 1.13 | 1.14 |
| Sample 16 | 1.03 | 1.02 | 1.11 |
| Sample 17 | 1.09 | 1.05 | 1.39 |
| Sample 18 | 1.35 | 1.28 | 1.46 |
| Sample 19 | 0.97 | 0.90 | 1.01 |
| Sample 20 | 1.20 | 1.19 | 1.08 |
| Sample 21 | 0.98 | 0.96 | 0.97 |
| Sample 22 | 1.41 | 1.31 | 1.46 |
| Sample 23 | 1.17 | 1.13 | 1.31 |
| Sample 24 | 1.29 | 1.54 | 1.38 |
| Sample 25 | 1.19 | 1.16 | 1.16 |
| Sample 26 | 0.98 | 1.18 | 0.96 |
| Sample 27 | 1.04 | 1.05 | 1.17 |
| Sample 28 | 0.97 | 1.07 | 1.12 |
| Sample 29 | 3.04 | 2.94 | 3.38 |
| Sample 30 | 1.19 | 1.32 | 1.49 |
| Sample 31 | 0.93 | 0.96 | 1.07 |
| Sample 32 | 1.05 | 1.03 | 1.20 |
| Sample 33 | 1.01 | 1.09 | 1.06 |
| Sample 34 | 1.31 | 1.44 | 1.46 |
| Sample 35 | 1.05 | 1.21 | 1.10 |
| Sample 36 | 1.03 | 1.20 | 1.12 |
| Sample 37 | 0.93 | 0.95 | 1.24 |
| Sample 38 | 1.00 | 0.96 | 1.12 |
| Sample 39 | 1.02 | 1.11 | 1.22 |
| Sample 40 | 1.05 | 1.27 | 1.82 |
| Sample 41 | 1.36 | 1.59 | 1.47 |
| Sample 42 | 2.15 | 2.23 | 1.28 |
| Sample 43 | 1.84 | 2.51 | 2.15 |
| Sample 44 | 1.10 | 1.32 | 1.08 |
| Sample 45 | 1.14 | 1.12 | 1.04 |
| Sample 46 | 1.26 | 1.30 | 1.65 |
| Sample 47 | 1.29 | 1.10 | 1.16 |
| Sample 48 | 1.09 | 1.19 | 1.17 |
| Sample 49 | 1.58 | 1.76 | 1.69 |
| Sample 50 | 0.93 | 1.14 | 1.07 |
| Sample 51 | 1.00 | 1.09 | 1.18 |
| Sample 52 | 1.00 | 1.03 | 1.31 |
| Sample 53 | 0.98 | 1.18 | 1.12 |
| Sample 54 | 0.92 | 1.21 | 1.00 |
| Sample 55 | 0.94 | 0.92 | 0.99 |
| Sample 56 | 0.94 | 0.99 | 1.16 |
| Sample 57 | 0.84 | 0.97 | 1.05 |
| Sample 58 | 0.91 | 0.94 | 1.07 |
| Sample 59 | 1.00 | 1.21 | 1.23 |
| Sample 60 | 1.02 | 1.06 | 1.18 |
| Sample 61 | 1.31 | 1.91 | 1.76 |
| Sample 62 | 1.03 | 1.17 | 1.30 |
| Sample 63 | 0.92 | 0.90 | 0.93 |
| Sample 64 | 0.93 | 0.99 | 1.15 |
| Sample 65 | 0.98 | 1.24 | 1.42 |
| Sample 66 | 1.36 | 1.44 | 1.15 |
| Sample 67 | 1.22 | 0.94 | 1.37 |
| Sample 68 | 0.98 | 1.00 | 1.11 |
| Sample 69 | 0.91 | 0.92 | 1.05 |
| Sample 70 | 1.05 | 1.33 | 1.32 |
| Sample 71 | 0.99 | 0.99 | 1.12 |
| Sample 72 | 1.35 | 1.74 | 1.87 |
| Sample 73 | 0.93 | 0.94 | 1.03 |
| Sample 74 | 0.89 | 1.09 | 1.04 |
| Sample 75 | 1.33 | 1.17 | 1.35 |
| Sample 76 | 1.33 | 1.57 | 1.80 |
| Sample 77 | 1.04 | 1.15 | 1.04 |
| Sample 78 | 2.79 | 2.92 | 2.99 |
| Sample 79 | 2.65 | 2.69 | 2.78 |
| Sample 80 | 0.96 | 0.91 | 1.07 |
| Sample 81 | 2.53 | 3.14 | 2.88 |
| Sample 82 | 1.15 | 1.21 | 1.15 |
| Sample 83 | 1.16 | 1.10 | 1.16 |
| Sample 84 | 1.81 | 1.37 | 1.82 |
| Sample 85 | 1.01 | 1.21 | 1.70 |
| Sample 86 | 1.65 | 1.95 | 1.73 |
| Sample 87 | 1.54 | 1.62 | 1.44 |
| Sample 88 | 1.13 | 1.13 | 1.23 |
| Sample 89 | 0.94 | 0.97 | 1.18 |
| Sample 90 | 1.00 | 0.98 | 1.04 |
| Sample 91 | 1.10 | 1.20 | 1.24 |
| Sample 92 | 1.10 | 1.15 | 1.18 |
| Sample 93 | 1.15 | 1.75 | 1.25 |
| Sample 94 | 1.14 | 1.22 | 1.21 |
| Sample 95 | 1.02 | 1.16 | 1.23 |
| Sample 96 | 1.16 | 1.20 | 1.23 |
| Sample 97 | 1.24 | 1.06 | 1.00 |
| Sample 98 | 1.31 | 1.69 | 1.76 |
| Sample 99 | 1.02 | 1.18 | 1.04 |
| Sample 100 | 0.92 | 1.01 | 0.96 |
| Sample 101 | 1.11 | 1.06 | 1.21 |
| Sample 102 | 1.08 | 1.20 | 1.32 |
| Sample 103 | 0.83 | 0.98 | 1.16 |
| Sample 104 | 1.02 | 1.09 | 0.99 |
| Sample 105 | 1.28 | 1.17 | 1.12 |
| Sample 106 | 1.16 | 1.17 | 1.13 |
| Sample 107 | 1.09 | 1.23 | 1.31 |
| Sample 108 | 2.16 | 1.59 | 2.07 |
| Sample 109 | 1.08 | 1.12 | 1.44 |
| Sample 110 | 1.11 | 1.22 | 1.53 |
| Sample 111 | 1.04 | 1.04 | 1.15 |
| Sample 112 | 0.89 | 1.19 | 1.19 |
| Sample 113 | 1.08 | 1.04 | 1.24 |
| Sample 114 | 1.05 | 1.20 | 1.42 |
| Sample 115 | 1.58 | 1.09 | 1.25 |
| Sample 116 | 1.08 | 1.09 | 1.35 |
| Sample 117 | 1.06 | 1.17 | 1.43 |
| Sample 118 | 1.39 | 1.23 | 1.66 |
| Sample 119 | 1.10 | 1.06 | 1.19 |
| Sample 120 | 1.61 | 1.68 | 1.65 |
| Sample 121 | 1.18 | 1.19 | 1.29 |
| Sample 122 | 2.30 | 2.38 | 2.47 |
| Sample 123 | 1.16 | 1.30 | 1.24 |
| Sample 124 | 1.07 | 1.16 | 1.17 |
| Sample 125 | 1.07 | 1.16 | 1.18 |
| Sample 126 | 1.29 | 1.47 | 1.83 |
| Sample 127 | 1.12 | 1.02 | 1.32 |
| Sample 128 | 1.08 | 2.21 | 1.64 |
| Sample 129 | 1.15 | 1.41 | 1.49 |
| Sample 130 | 0.99 | 0.98 | 1.13 |
| Sample 131 | 1.21 | 1.41 | 1.38 |
| Sample 132 | 0.92 | 0.92 | 1.00 |
| Sample 133 | 1.13 | 1.23 | 1.26 |
| Sample 134 | 1.00 | 1.06 | 1.15 |
| Sample 135 | 0.96 | 1.23 | 2.12 |
| Sample 136 | 2.02 | 1.81 | 1.37 |
| Sample 137 | 2.85 | 3.03 | 2.89 |
| Sample 138 | 1.01 | 1.50 | 1.55 |
| Sample 139 | 2.55 | 2.84 | 2.82 |
| Sample 140 | 0.89 | 1.18 | 1.32 |
| Sample 141 | 0.90 | 1.18 | 1.13 |
| Sample 142 | 1.20 | 1.18 | 1.31 |
| Sample 143 | 1.02 | 1.14 | 1.27 |
| Sample 144 | 0.90 | 1.02 | 1.08 |
| Sample 145 | 1.31 | 1.16 | 1.38 |
| Sample 146 | 1.29 | 1.41 | 1.31 |
| Sample 147 | 1.21 | 1.16 | 1.12 |
| Sample 148 | 1.37 | 1.30 | 1.21 |
| Sample 149 | 0.91 | 1.30 | 2.99 |
| Sample 150 | 1.10 | 1.29 | 1.68 |
| Sample 151 | 0.95 | 1.27 | 1.28 |
| Sample 152 | 0.92 | 1.16 | 1.98 |
| Sample 153 | 0.97 | 1.00 | 1.01 |
| Sample 154 | 0.93 | 0.92 | 1.06 |
| Sample 155 | 1.25 | 1.24 | 1.27 |
| Sample 156 | 1.45 | 1.84 | 1.44 |
| Sample 157 | 1.57 | 1.48 | 1.61 |
| Sample 158 | 1.08 | 1.20 | 1.25 |
| Sample 159 | 1.11 | 1.20 | 1.17 |
| Sample 160 | 1.37 | 1.42 | 1.13 |
| Sample 161 | 0.89 | 1.17 | 1.31 |
| Sample 162 | 0.93 | 1.00 | 1.46 |
| Sample 163 | 0.99 | 1.14 | 1.20 |
| Sample 164 | 1.21 | 1.03 | 1.10 |
| Sample 165 | 1.24 | 1.45 | 1.61 |
| Sample 166 | 0.97 | 1.20 | 1.39 |
| Sample 167 | 0.95 | 0.92 | 0.96 |
| Sample 168 | 1.07 | 1.08 | 1.01 |
| Sample 169 | 1.10 | 1.31 | 1.34 |
| Sample 170 | 0.87 | 1.28 | 1.10 |
| Sample 171 | 0.94 | 0.94 | 1.01 |
| Sample 172 | 0.85 | 1.19 | 1.24 |
| Sample 173 | 1.58 | 1.68 | 1.55 |
| Sample 174 | 0.97 | 0.87 | 0.97 |
| Sample 175 | 0.97 | 1.01 | 1.12 |
| Sample 176 | 1.61 | 0.94 | 1.24 |
| Sample 177 | 1.42 | 1.37 | 1.44 |
| Sample 178 | 1.06 | 1.31 | 0.88 |
| Sample 179 | 2.80 | 2.73 | 1.25 |
| Sample 180 | 1.09 | 1.03 | 1.30 |
| Sample 181 | 1.02 | 1.05 | 1.16 |
| Sample 182 | 1.11 | 1.28 | 1.25 |
| Sample 183 | 1.02 | 1.00 | 1.16 |
| Sample 184 | 1.48 | 1.22 | 1.17 |
| Sample 185 | 1.13 | 1.30 | 1.25 |
| Sample 186 | 1.22 | 1.15 | 1.09 |
| Sample 187 | 1.38 | 1.32 | 1.42 |
| Sample 188 | 4.01 | 2.98 | 1.38 |
| Sample 189 | 1.42 | 1.21 | 0.98 |
| Sample 190 | 0.96 | 0.96 | 1.17 |
| Sample 191 | 2.79 | 2.82 | 3.95 |

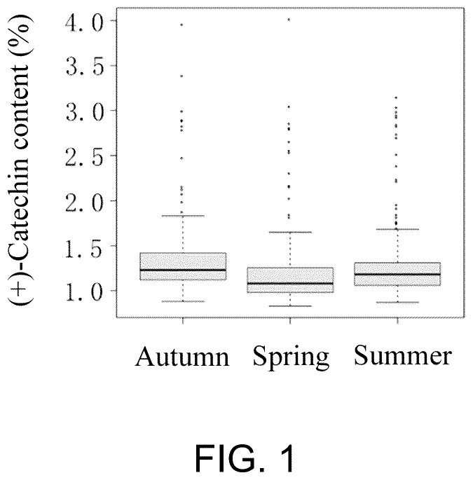

The variation of (+)-catechin content in the population is shown in Table 2 and FIG. 1.

TABLE 2
Phenotypic variation in (+)-catechin traits

| | | | Standard | Coefficient of | Diversity | |
|---|---|---|---|---|---|---|
| Season | Range (%) | Mean (%) | deviation ^{a}SD | variation ^{b}CV | index ^{C}H′ | Heritability |
| Spring | 0.83~4.01 | 1.22 | 0.45 | 0.37 | 1.49 | 0.90 |
| Summer | 0.87~3.14 | 1.3 | 0.44 | 0.34 | 1.51 | |
| Autumn | 0.88~3.95 | 1.36 | 0.44 | 0.32 | 1.58 | |

### III. Association Analysis Between Genotype and Traits

### 1. Experimental Procedure

The CTAB method was used to extract total DNA from buds of 191 tea plant resources, and it was ensured that A260/A280 of each DNA sample is between 1.8 and 2.0, and the concentration was greater than 100 μg/μl. The extracted DNA samples were used to detect genotypes located in the SNP site 1 (Scaffold4239:309117), the SNP site 2 (Scaffold3614: 66549), the SNP site 3 (Scaffold349: 3413816), the SNP site 4 (Scaffold1989: 2316385), the SNP site 5 (Scaffold451: 940283), the SNP site 6 (Scaffold3727:442660), the SNP site 7 (Scaffold115:803980), and the SNP site 8 (Scaffold920:281727) of the “Shuchazao” CSS cultivar tea plant genome (http://tpia.teaplant.org/index.html), respectively. The association analysis of traits and markers was performed, significance level of the association was judged by P-value, and the p-value less than 1.25E−05 was the significance level.

### 2. Experimental Results

The p-values of the eight SNP sites in different seasons are shown in Table 3.

TABLE 3
p-values of eight SNP sites in different seasons

Season

| | Spring | Summer | Autumn |
|---|---|---|---|
| Scaffold4239: 309117 | 2.03E−08 | 5.94E−08 | 1.48E−07 |
| Scaffold3614: 66549 | 3.75E−16 | 2.98E−19 | 5.46E−15 |
| Scaffold349: 3413816 | 3.54E−13 | 5.96E−15 | 2.43E−13 |
| Scaffold1989: 2316385 | 2.67E−15 | 1.68E−19 | 1.80E−15 |
| Scaffold451: 940283 | 3.14E−06 | 2.42E−06 | 2.19E−06 |
| Scaffold3727: 442660 | 5.49E−07 | 3.18E−08 | 4.49E−07 |
| Scaffold115: 803980 | 1.23E−13 | 9.83E−14 | 1.83E−10 |
| Scaffold920: 281727 | 8.97E−21 | 3.13E−21 | 8.26E−12 |

### Embodiment 2 Verification of SNP Site

### I. Experimental Method

Genotypes of the SNP site 1 (Scaffold4239:309117), the SNP site 2 (Scaffold3614: 66549), the SNP site 3 (Scaffold349: 3413816), the SNP site 4 (Scaffold1989: 2316385), the SNP site 5 (Scaffold451: 940283), the SNP site 6 (Scaffold3727:442660), the SNP site 7 (Scaffold115:803980), and the SNP site 8 (Scaffold920:281727) were subjected to verification in another population of 98 germplasms.

1. (+)-Catechin content of each sample was detected. The specific detection method is the same as that of Embodiment 1.

2. SnapShot technology platform was used to detect the genotypes of the SNP site 1 (Scaffold4239:309117), the SNP site 2 (Scaffold3614: 66549), the SNP site 3 (Scaffold349: 3413816), the SNP site 4 (Scaffold1989: 2316385), the SNP site 5 (Scaffold451: 940283), the SNP site 6 (Scaffold3727:442660), the SNP site 7 (Scaffold115:803980), and the SNP site 8 (Scaffold920:281727).

This method designed primers of different lengths for different mutation sites, after SNAPshot reaction, the products were analyzed by electrophoresis, five-color fluorescence detection, and Gene mapper analysis, and multiple SNP sites can be detected in one sequencing reaction. SNAPshot was used for site-specific sequence analysis, and the basic principle thereof followed the dideoxy termination method in direct DNA sequencing, except that only ddNTPs with different fluorescent labels were used in the PCR reaction. Since the 3′-end of the primers of each SNP site is close to the SNP point, each of the primers was extended by only one nucleotide according to the sequence of the template under the action of the polymerase. Then an advanced fluorescence detection system was used to detect the type of that nucleotide that is extended.

### (1) Design of Primers

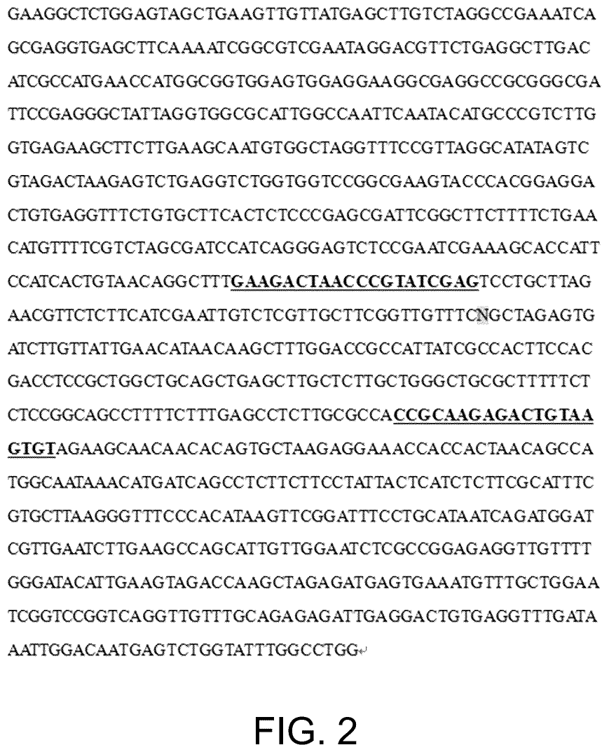

Primers were designed and synthesized according to the position of Scaffold4239:309117. In particular, Scaffold4239:309117 each extended 500 bp upstream and downstream. A nucleotide sequence thereof is shown as SEQ ID NO: 1 (FIG. 2, wherein N denotes the base to be tested at Scaffold4239:309117).

PCR primers:

| (SEQ ID NO: 2) | |
|---|---|
| F: | GAAGACTAACCCGTATCGAG; |
| (SEQ ID NO: 3) | |
| R: | ACACTTACAGTCTCTTGCGG. |

Single base extension primer:

ctgactgactgactgactgactATTGTCTCGTTGCTTCGGTTGTTTC.

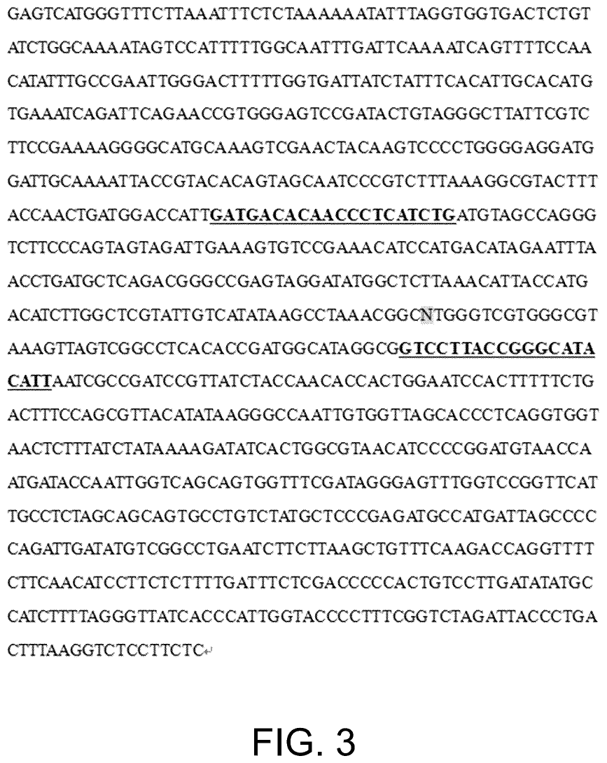

Primers were designed and synthesized according to the position of Scaffold3614: 66549. In particular, Scaffold3614: 66549 each extended 500 bp upstream and downstream. A nucleotide sequence thereof is shown as SEQ ID NO: 5 (FIG. 3, wherein N denotes the base to be tested at Scaffold3614: 66549).

PCR primers:

F:
(SEQ ID NO: 5)
GATGACACAACCCTCATCTG;
R:
(SEQ ID NO: 6)
AATGTATGCCCGGTAAGGAC.

Single base extension primer:

gactACTAACTTTACGCCCACGACCCA.

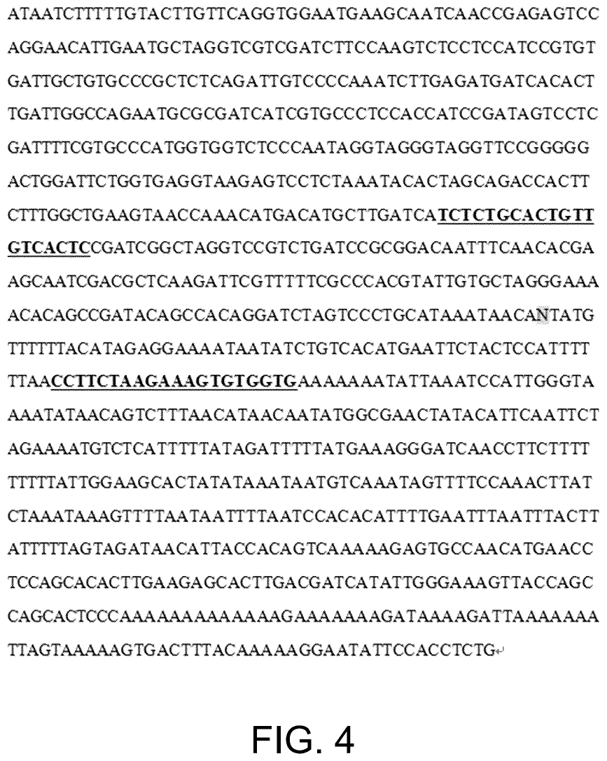

Primers were designed and synthesized according to the position of Scaffold349: 3413816. In particular, Scaffold349: 3413816 each extended 500 bp upstream and downstream. A nucleotide sequence thereof is shown as SEQ ID NO: 7 (FIG. 4, wherein N denotes the base to be tested at Scaffold349: 3413816).

PCR primers:

primer F:
(SEQ ID NO: 8)
TCTCTGCACTGTTGTCACTC;
primer R:
(SEQ ID NO: 9)
CACCACACTTTCTTAGAAGG.

Single base extension primer:

actgactgactaAGGATCTAGTCCCTGCATAAATAACA.

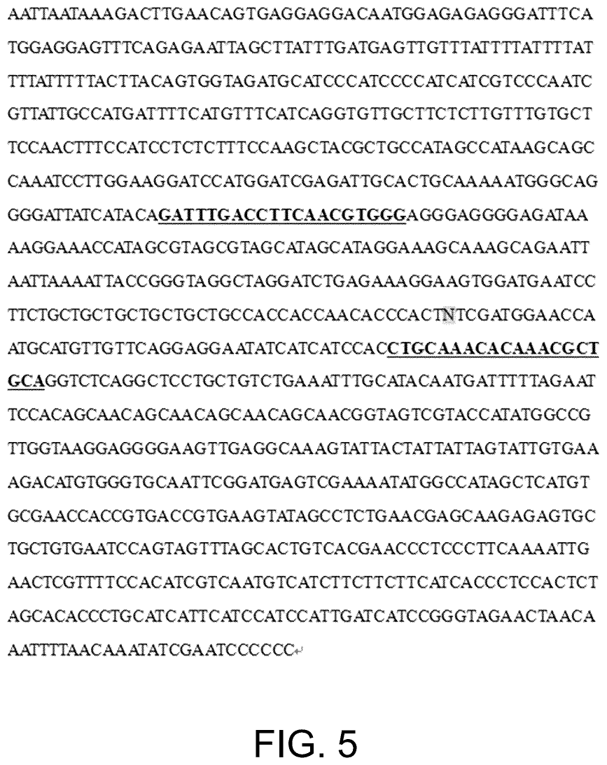

Primers were designed and synthesized according to the position of Scaffold1989: 2316385. In particular, Scaffold1989: 2316385 each extended 500 bp upstream and downstream. A nucleotide sequence thereof is shown as SEQ ID NO: 10 (FIG. 5, wherein N denotes the base to be tested at Scaffold1989: 2316385).

PCR primers:

primer F:
(SEQ ID NO: 11)
GATTTGACCTTCAACGTGGG;
primer R:
(SEQ ID NO: 12)
TGCAGCGTTTGTGTTTGCAG.

Single base extension primer:

CTGCTGCCACCACCAACACCCACT.

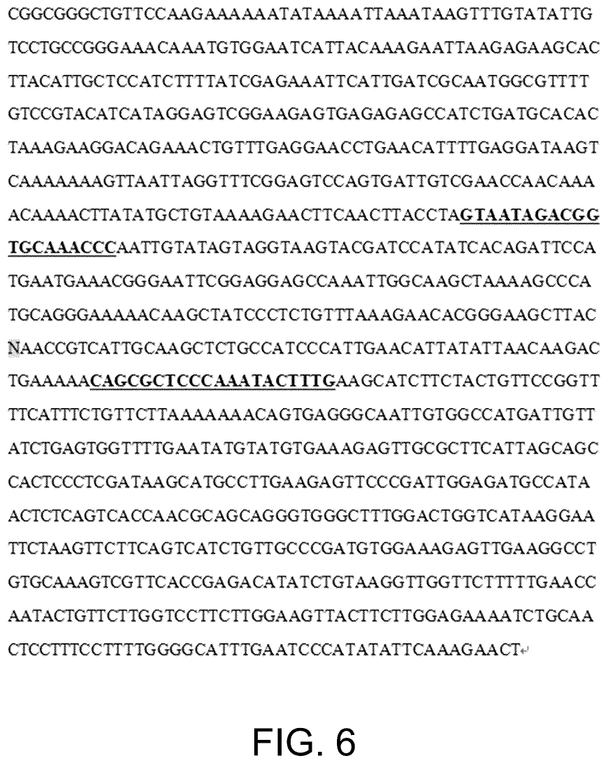

Primers were designed and synthesized according to the position of Scaffold451: 940283. In particular, Scaffold451: 940283 each extended 500 bp upstream and downstream. A nucleotide sequence thereof is shown as SEQ ID NO: 13 (FIG. 6, wherein N denotes the base to be tested at Scaffold451: 940283).

PCR primers:

F:
(SEQ ID NO: 14)
GTAATAGACGGTGCAAACCC;
R:
(SEQ ID NO: 15)
CAAAGTATTTGGGAGCGCTG.

Single base extension primer:

actgactGTTTAAAGAACACGGGAAGCTTAC.

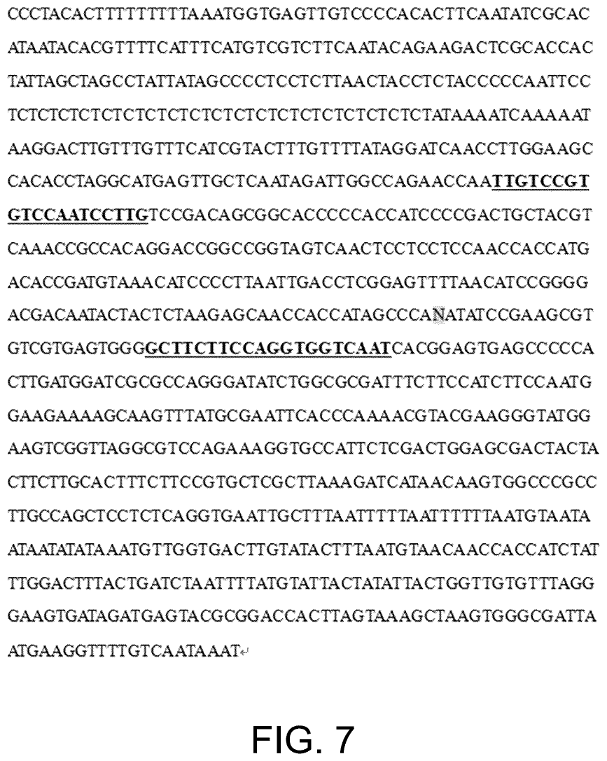

Primers were designed and synthesized according to the position of Scaffold3727:442660. In particular, Scaffold3727:442660 each extended 500 bp upstream and downstream. A nucleotide sequence thereof is shown as SEQ ID NO: 16 (FIG. 7, wherein N denotes the base to be tested at Scaffold3727:442660).

PCR primers:

F:
(SEQ ID NO: 17)
TTGTCCGTGTCCAATCCTTG;
R:
(SEQ ID NO: 16)
ATTGACCACCTGGAAGAAGC.

Single base extension primer:

ataaTCTAAGAGCAACCACCATAGCCCA.

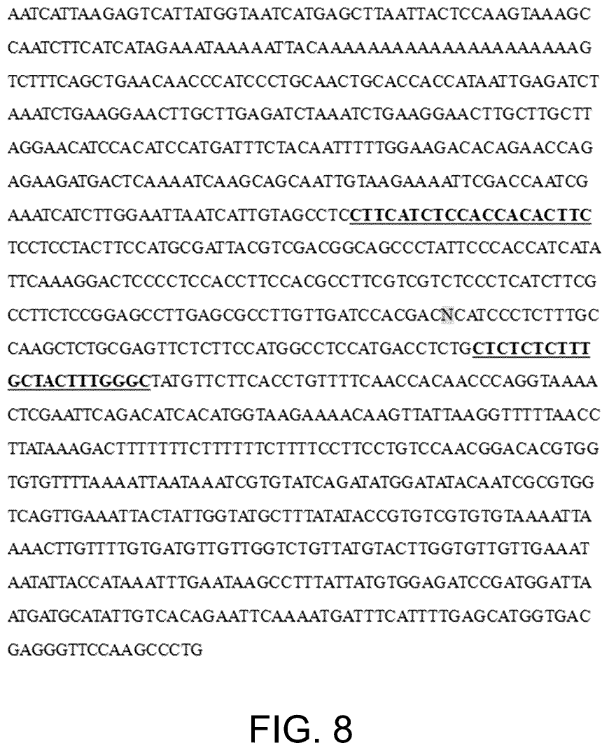

Primers were designed and synthesized according to the position of Scaffold115: 803980. In particular, Scaffold115: 803980 each extended 500 bp upstream and downstream. A nucleotide sequence thereof is shown as SEQ ID NO: 19 (FIG. 8, wherein N denotes the base to be tested at Scaffold115: 803980).

PCR primers:

F:
(SEQ ID NO: 20)
CTTCATCTCCACCACACTTC;
R:
(SEQ ID NO: 21)
GCCCAAAGTAGCAAAGAGAG.

Single base extension primer:

gactgactgactgactgactgactcaGCAGAGCTTGGCAAAGAGGGATG.

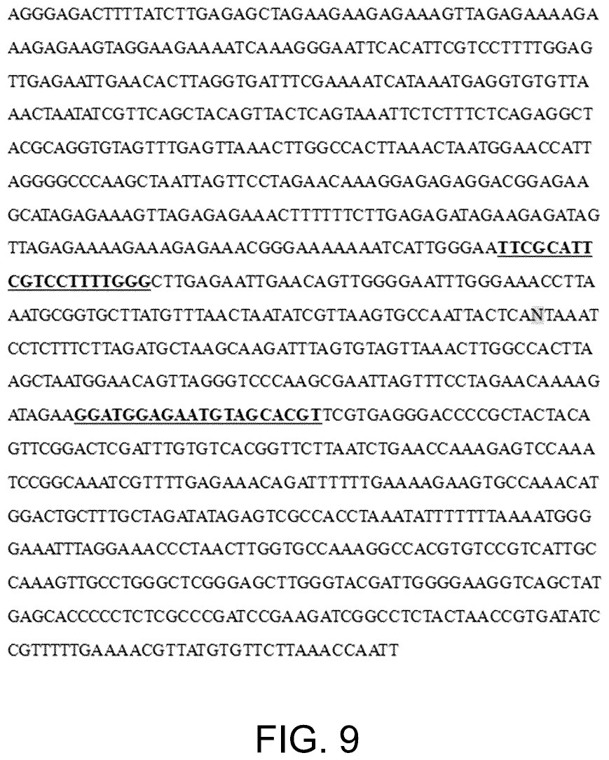

Primers were designed and synthesized according to the position of Scaffold920: 281727. In particular, Scaffold920: 281727 each extended 500 bp upstream and downstream. A nucleotide sequence thereof is shown as SEQ ID NO: 22 (FIG. 9, wherein N denotes the base to be tested at Scaffold920: 281727).

PCR primers:

primer F:
(SEQ ID NO: 23)
TTCGCATTCGTCCTTTTGGG;
primer R:
(SEQ ID NO: 24)
ACGTGCTACATTCTCCATCC.

Single base extension primer:

tgactgactgactgactgactgactgactgactTAGCATCTAAGAAAGAG
GATTTA.

(2) PCR Amplification

PCR system (10 μl) was as follows:

| | |
|---|---|
| 2 × Taq PCR Master Mix | 5 μl |
| PrimerMix (matching according | 1 μl |
| to the amplification ratio) | |
| DNA template | 1 μl |
| ddH₂O | 3 μl |

PCR amplification procedure was as follows:

| | | |
|---|---|---|
| 95° C. | 5 minutes | |
| 95° C. | 30 seconds | ×45 cycles |
| 56° C. | 30 seconds | |
| 72° C. | 30 seconds | |
| 72° C. | 2 minutes | |
| 4° C. | forever | |

### (3) PCR Product Purification

Purification was performed using shrimp alkaline phosphatase purification. The main functional components of shrimp alkaline phosphatase MIX (EX-SAP) are SAP and ExoI.SAP enzyme, which can dephosphorylate residual dNTPs, and Exol degrades the free single-chain primer. 4 μl of PCR product was taken and added with 2 μl of EX-SAP enzyme. The specific reaction system is shown as follows:

| Constituent of digestive system | Volume (μl) |
|---|---|
| ddH₂O | 0.75 |
| SAP (1U/μl) | 0.5 |
| ExoI (5U/μl) | 0.15 |
| 10*SAP buffer | 0.6 |
| PCR product | 4 |
| Total volume | 6 |

After that, digestion and incubation were performed on a PCR instrument: 37° C. for 40 minutes, 85° C. for 5 minutes, 4° C. forever.

### (4) SNAPshot Reaction

The PCR product was used as a template for SNAPshot reaction.

The SNAPshot reaction system is shown as follows:

| Reagent | Dosage (μl) |
|---|---|
| SNaPshot Mix | 0.5 |
| Pooled PCR Products | 3 |
| Pooled Primers | 1 |
| dH₂O | 0.5 |
| Total volume | 5 |

The SNAPshot reaction procedure is:

| | | |
|---|---|---|
| 95° C. | 2 minutes | |
| 95° C. | 10 seconds | ×40 cycles |
| 52° C. | 5 seconds | |
| 60° C. | 30 seconds | |
| 4° C. | forever | |

After that, the SNAPshot product was purified, and 2 μl of SAP mix was directly added to the SNAPshot reaction product. The specific reaction system was as follows:

| Constituent | Volume (μl) |
|---|---|
| Water | 0.9 |
| SAP(1U/ul) | 0.5 |
| 10*SAP buffer | 0.6 |
| Total | 2 |

The SNAPshot product digestion reaction was performed on a PCR instrument, and the reaction procedures were: 37° C. for 40 minutes, 75° C. for 15 minutes, 4° C. forever.

### (5) On-Machine Detection

2 μl of the digested SNAPshot reaction product was taken and added into 8 μl of deionized formamide containing 0.4% LIZ120, denatured at 95° C. for 5 minutes, then quenched at −20° C., and then sequenced on 3730XL.

### (6) Result Analysis

The .fsa results obtained by GeneMarker analysis were used to derive peak plots and table files, and to calculate the SNP mutant type of each sample.

### II. Experimental Results

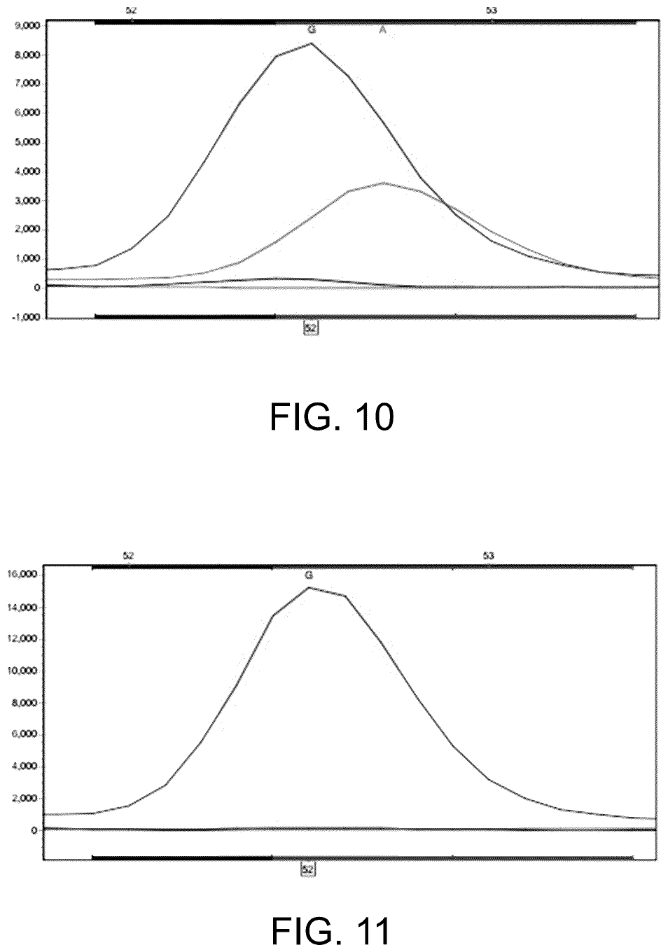

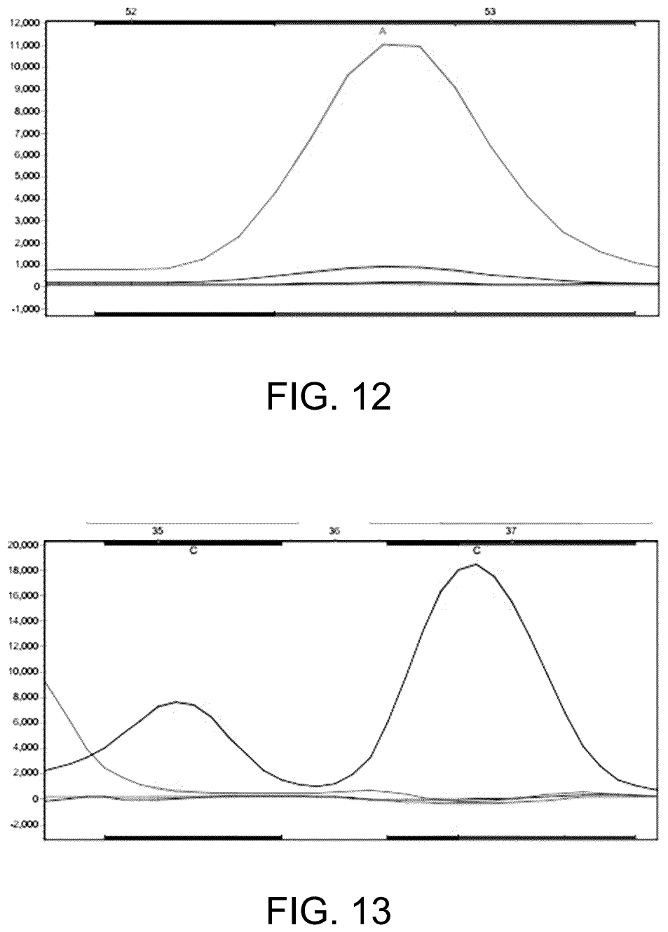

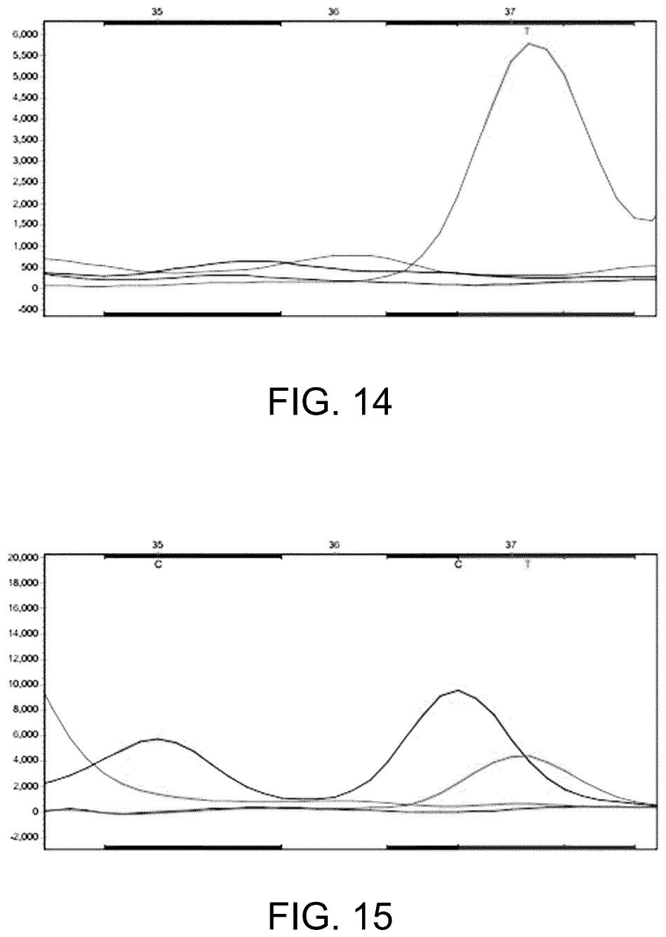

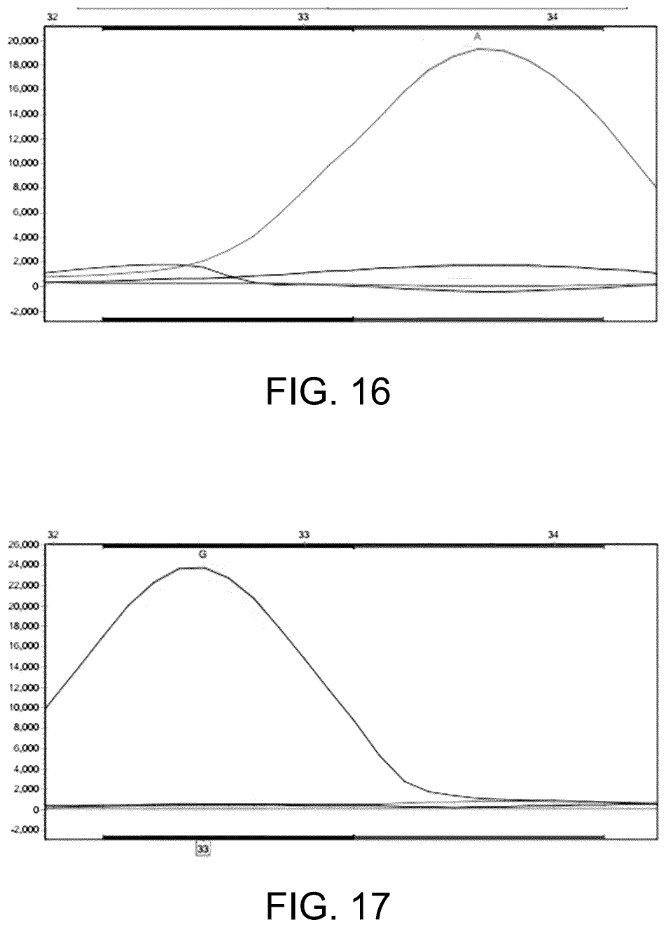

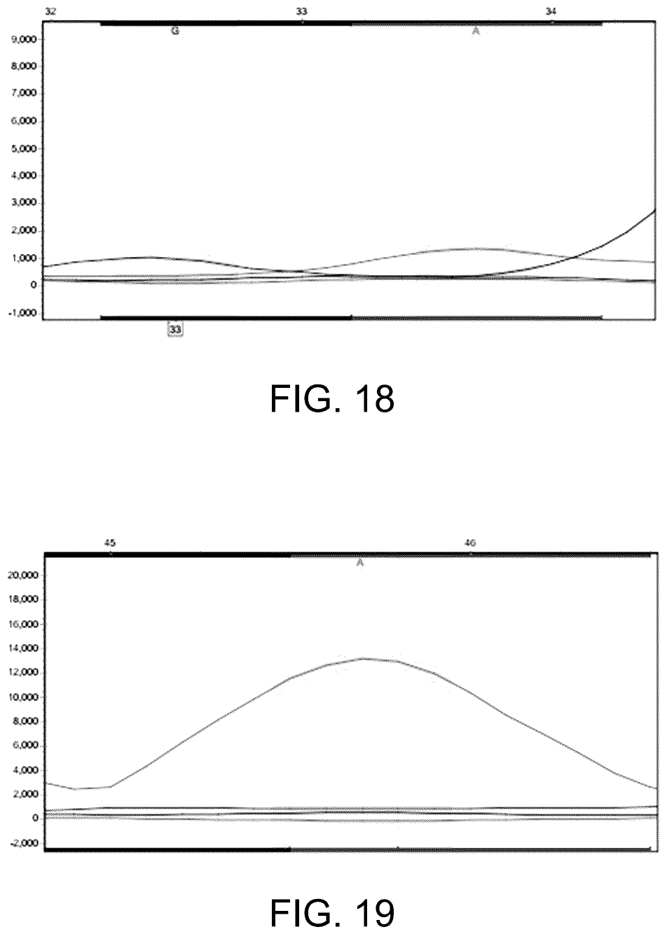

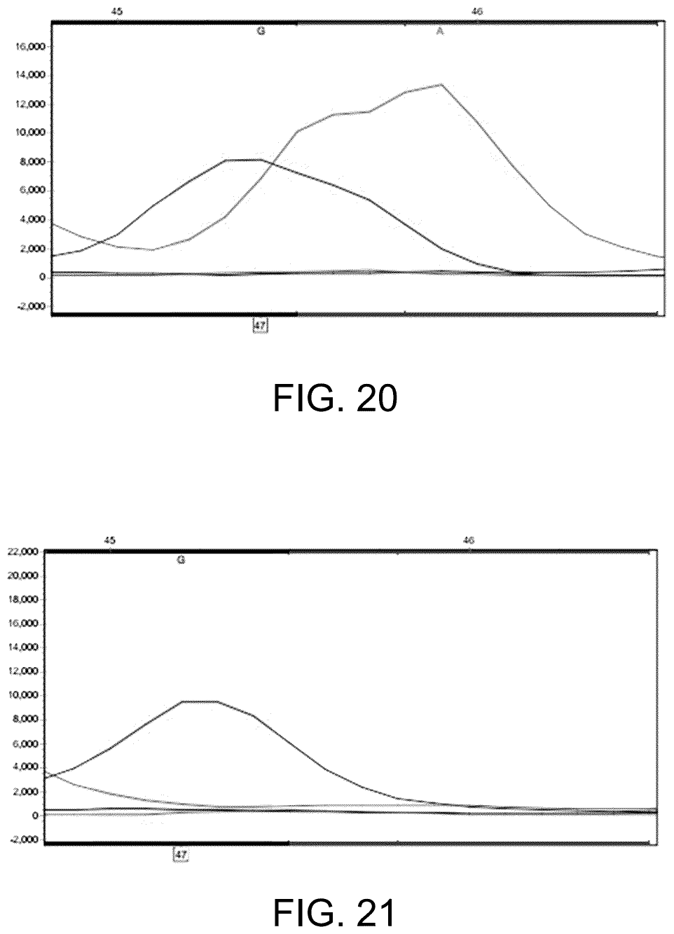

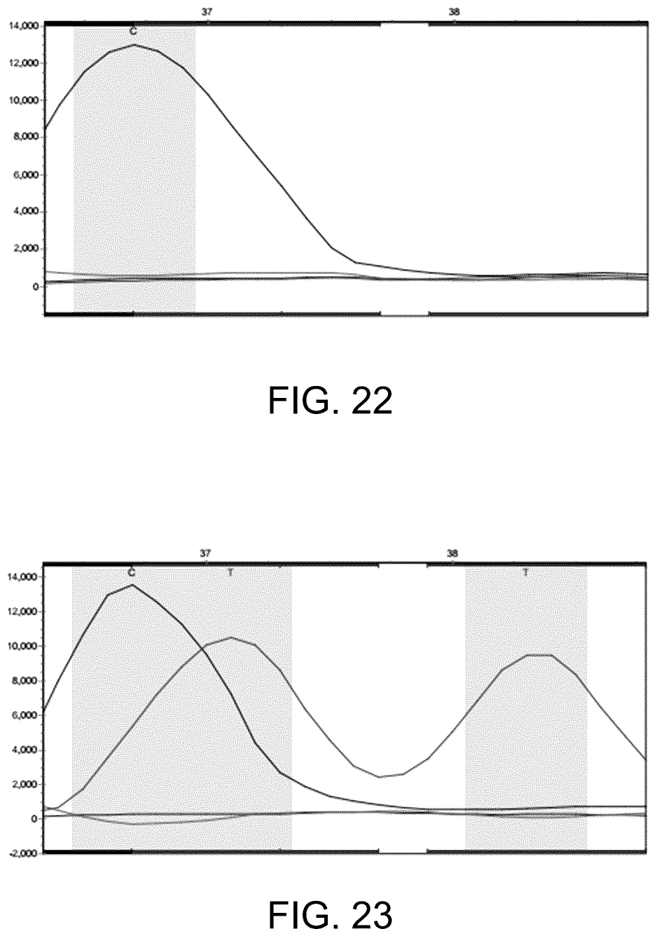

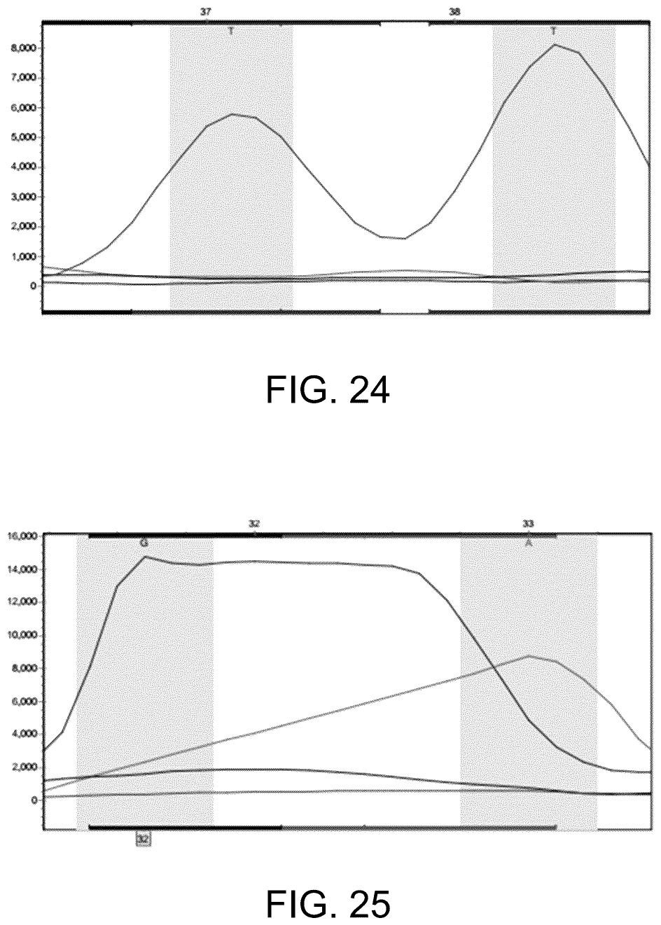

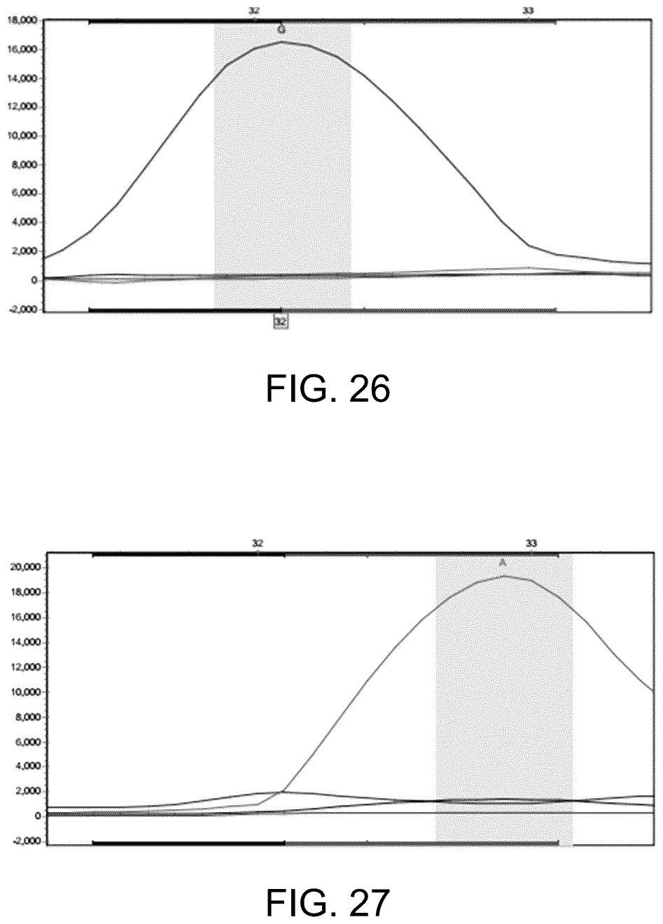

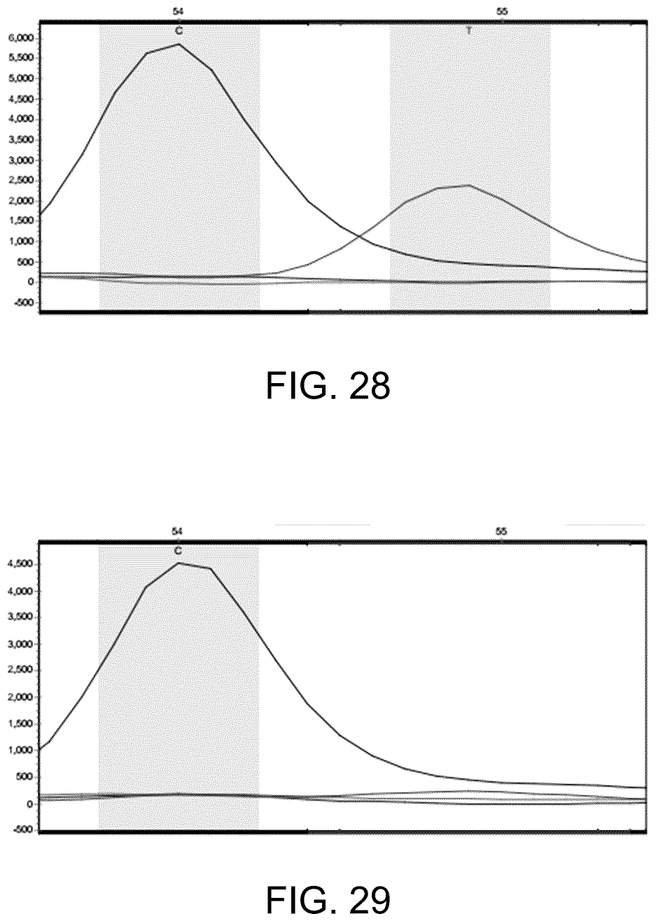

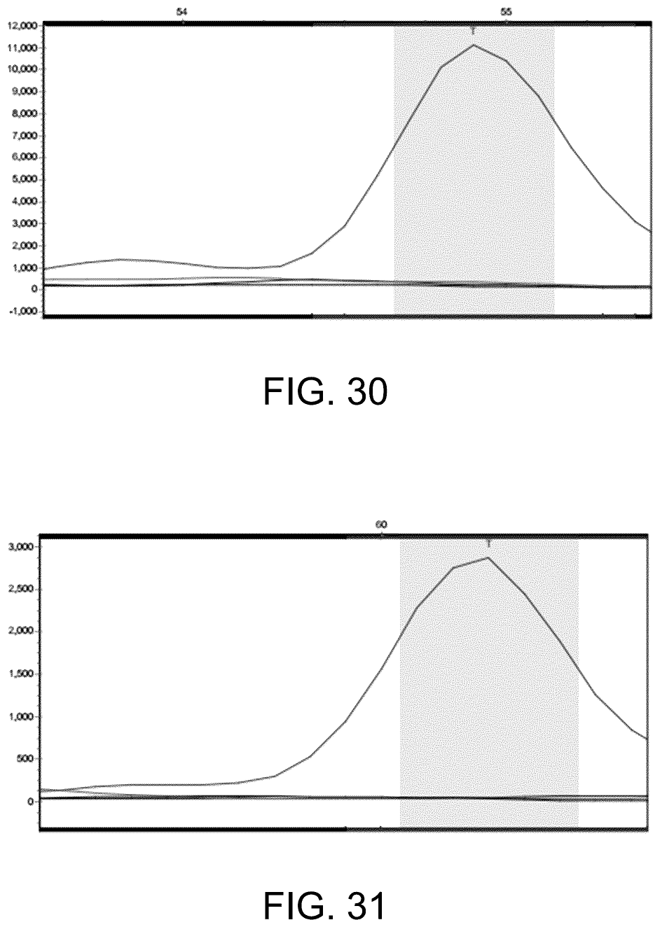

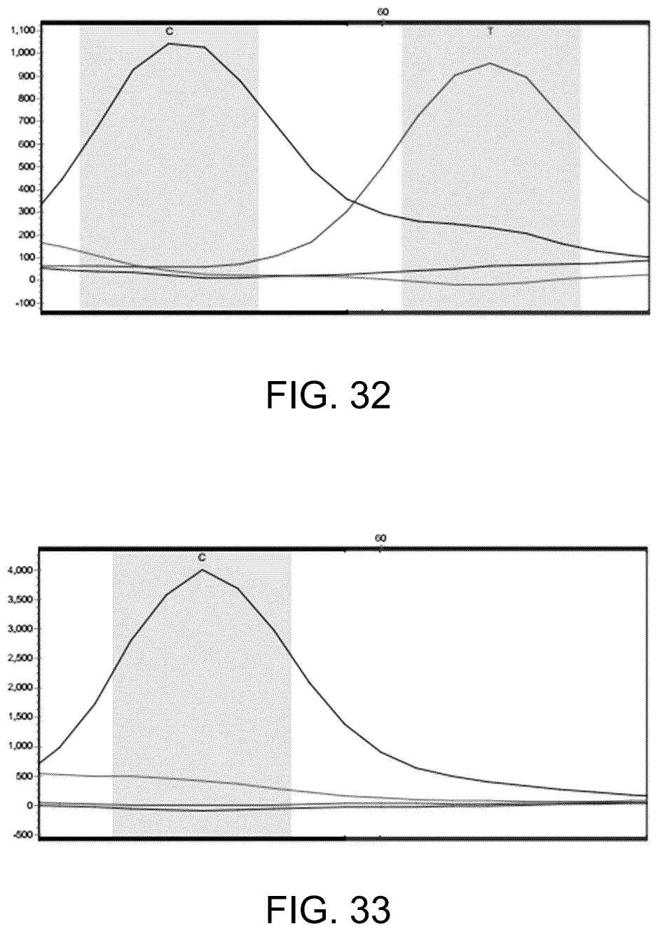

(+)-Catechin content and genotypes of SNP1, SNP2, SNP3, SNP4, SNP5, SNP6, SNP7, SNP8 sites of each sample are shown in Table 4, and the SNAPshot sequencing results of some samples are shown in FIG. 8 to FIG. 24.

TABLE 4
The (+)-catechin content in dry matter and genotype of the resource in the population:

| | (+)-Catechin | SNP1 | SNP2 | SNP3 | SNP4 | SNP5 | SNP6 | SNP7 | SNP8 |
|---|---|---|---|---|---|---|---|---|---|
| Sample | content (%) | genotype | genotype | genotype | genotype | genotype | genotype | genotype | genotype |
| Sample 2-1 | 1.00 | GA | CT | AA | GG | CC | GA | AA | AA |
| Sample 2-2 | 0.98 | GA | CC | AA | AA | CC | GA | AA | AA |
| Sample 2-3 | 0.99 | GG | TT | AA | GG | CC | GG | AA | AA |
| Sample 2-4 | 1.08 | GG | TT | AA | GG | CC | GG | AA | AA |
| Sample 2-5 | 0.91 | GG | CT | AA | GG | CC | GG | AA | AA |
| Sample 2-6 | 1.18 | GG | TT | AA | GG | CC | GG | AA | AA |
| Sample 2-7 | 1.12 | GG | TT | AA | GG | CC | GG | AA | AA |
| Sample 2-8 | 0.88 | GG | TT | AA | GG | CC | GG | AA | AA |
| Sample 2-9 | 0.89 | GG | TT | AA | GG | CC | GG | AA | AA |
| Sample 2-10 | 1.07 | GG | TT | AA | GG | CC | GG | AA | AA |
| Sample 2-11 | 1.10 | AA | CT | AA | GG | CC | AA | AA | AA |
| Sample 2-12 | 0.90 | GG | TT | GA | GG | CC | GG | AA | AA |
| Sample 2-13 | 1.09 | GA | CT | AA | GG | CC | GA | AA | AA |
| Sample 2-14 | 3.44 | AA | CC | AA | GG | CC | AA | AA | AA |
| Sample 2-15 | 1.10 | GG | TT | AA | GG | CC | GG | AA | AA |
| Sample 2-16 | 0.96 | GG | TT | AA | GG | CC | GG | AA | AA |
| Sample 2-17 | 0.99 | GG | TT | AA | GG | CC | GG | AA | AA |
| Sample 2-18 | 1.07 | AA | CT | AA | GG | CC | AA | AA | AA |
| Sample 2-19 | 1.18 | GG | TT | GA | GG | CC | GG | AA | AA |
| Sample 2-20 | 1.95 | AA | CT | AA | GG | CC | AA | AA | AA |
| Sample 2-21 | 0.99 | GG | TT | AA | GG | CC | GG | AA | AA |
| Sample 2-22 | 1.00 | GG | TT | AA | GG | CC | GG | AA | Not detected |
| Sample 2-23 | 0.98 | GG | TT | AA | GG | CC | GG | AA | AA |
| Sample 2-24 | 1.12 | GA | CT | AA | GG | CC | GA | AA | AA |
| Sample 2-25 | 1.11 | GG | TT | AA | GG | CC | GG | AA | AA |
| Sample 2-26 | 1.18 | GA | CT | AA | GG | CC | GA | AA | AA |
| Sample 2-27 | 1.03 | GA | TT | AA | GG | CC | GA | GA | AA |
| Sample 2-28 | 0.96 | GG | TT | AA | GG | CC | GG | AA | Not detected |
| Sample 2-29 | 0.98 | GG | TT | AA | GG | CC | GG | AA | AA |
| Sample 2-30 | 0.98 | GG | TT | AA | GG | CC | GG | AA | AA |
| Sample 2-31 | 0.96 | GG | TT | AA | GG | CC | GG | AA | AA |
| Sample 2-32 | 1.01 | GG | TT | GA | GG | CC | GG | AA | Not detected |
| Sample 2-33 | 0.81 | GG | TT | AA | GG | CC | GG | AA | AA |
| Sample 2-34 | 1.06 | GG | TT | AA | GG | CC | GG | AA | AA |
| Sample 2-35 | 1.13 | GG | TT | AA | GG | CC | GG | AA | AA |
| Sample 2-36 | 0.92 | GG | TT | GA | GG | CC | GG | AA | AA |
| Sample 2-37 | 1.25 | GA | TT | AA | GG | CC | GA | AA | AA |
| Sample 2-38 | 0.97 | GG | CC | GA | GG | CC | GG | AA | Not detected |
| Sample 2-39 | 0.99 | GA | TT | AA | GG | CC | GA | AA | AA |
| Sample 2-40 | 0.93 | GA | TT | AA | GG | CC | GA | AA | AA |
| Sample 2-41 | 0.87 | GG | TT | AA | GG | CC | GG | AA | AA |
| Sample 2-42 | 0.99 | GG | TT | AA | GG | CC | GG | AA | AA |
| Sample 2-43 | 1.16 | GA | TT | AA | GG | CC | GA | AA | AA |
| Sample 2-44 | 1.18 | AA | CT | AA | GG | CC | AA | AA | AA |
| Sample 2-45 | 1.20 | GG | TT | GA | GG | CC | GG | AA | Not detected |
| Sample 2-46 | 1.01 | GA | TT | AA | GG | CC | GA | GA | AA |
| Sample 2-47 | 0.92 | GG | TT | AA | GG | CC | GG | AA | AA |
| Sample 2-48 | 0.96 | GG | TT | AA | GG | CC | GG | AA | Not detected |
| Sample 2-49 | 1.02 | GG | TT | GA | GG | CC | GG | AA | AA |
| Sample 2-50 | 0.97 | GG | TT | AA | GG | CC | GG | GA | AA |
| Sample 2-51 | 0.89 | GA | TT | AA | GG | CC | GA | AA | AA |
| Sample 2-52 | 1.13 | GG | TT | AA | GG | CC | GG | AA | AA |
| Sample 2-53 | 1.21 | GG | TT | AA | GG | CC | GG | AA | AA |
| Sample 2-54 | 1.12 | GG | TT | AA | GG | CC | GG | AA | AA |
| Sample 2-55 | 1.11 | GG | TT | AA | GG | CC | GG | AA | AA |
| Sample 2-56 | 1.02 | GG | TT | AA | GG | CC | GG | AA | AA |
| Sample 2-57 | 0.99 | GG | TT | AA | GA | CC | GG | AA | AA |
| Sample 2-58 | 1.03 | GG | TT | AA | GG | CC | GG | AA | AA |
| Sample 2-59 | 1.14 | GG | TT | AA | GG | CC | GG | AA | AA |
| Sample 2-60 | 1.04 | GG | TT | AA | GG | CC | GG | AA | AA |
| Sample 2-61 | 0.97 | GG | TT | AA | GG | CC | GG | AA | AA |
| Sample 2-62 | 1.09 | GG | TT | AA | GG | CC | GG | AA | AA |
| Sample 2-63 | 1.45 | AA | TT | AA | GG | CC | AA | AA | AA |
| Sample 2-64 | 0.96 | GG | TT | AA | GA | CC | GG | AA | AA |
| Sample 2-65 | 1.09 | GG | TT | AA | GG | CC | GG | AA | Not detected |
| Sample 2-66 | 1.24 | GG | TT | GG | GG | CC | GG | AA | AA |
| Sample 2-67 | 1.05 | GG | TT | AA | GG | CC | GG | AA | AA |
| Sample 2-68 | 0.89 | GG | TT | GA | GG | CC | GG | AA | AA |
| Sample 2-69 | 0.97 | GG | CT | AA | GA | CC | GG | AA | AA |
| Sample 2-70 | 1.05 | GG | TT | AA | GG | CC | GG | AA | Not detected |
| Sample 2-71 | 1.09 | GG | TT | AA | GG | CC | GG | AA | AA |
| Sample 2-72 | 0.95 | GA | CT | AA | GG | CC | GA | AA | AA |
| Sample 2-73 | 1.10 | GA | CT | AA | GG | CC | GA | AA | AA |
| Sample 2-74 | 1.13 | GG | TT | AA | GG | CC | GG | AA | Not detected |
| Sample 2-75 | 1.25 | AA | CT | AA | GG | CC | AA | AA | AA |
| Sample 2-76 | 1.04 | GG | TT | AA | GG | CC | GG | AA | AA |
| Sample 2-77 | 0.97 | GA | CC | GG | AA | CT | GA | GA | GG |
| Sample 2-78 | 0.88 | GG | TT | AA | GA | CC | GG | AA | AA |
| Sample 2-79 | 1.28 | GG | TT | AA | GG | CC | GG | AA | AA |
| Sample 2-80 | 0.89 | GA | TT | AA | GG | CC | GA | GA | AA |
| Sample 2-81 | 0.92 | GG | TT | AA | GG | CC | GG | AA | AA |
| Sample 2-82 | 1.01 | GG | TT | AA | GG | CC | GG | AA | AA |
| Sample 2-83 | 1.08 | GA | TT | AA | GG | CC | GA | AA | AA |
| Sample 2-84 | 1.24 | GG | TT | AA | GG | CC | GG | AA | AA |
| Sample 2-85 | 0.97 | GG | CT | AA | GG | CC | GG | AA | AA |
| Sample 2-86 | 0.98 | GA | TT | AA | GG | CC | GA | AA | AA |
| Sample 2-87 | 1.03 | GA | TT | AA | GG | CC | GA | AA | AA |
| Sample 2-88 | 1.15 | GA | TT | AA | GG | CC | GA | AA | AA |
| Sample 2-89 | 0.98 | GA | TT | AA | GG | CC | GA | AA | AA |
| Sample 2-90 | 0.86 | GG | TT | AA | GG | CC | GG | AA | AA |
| Sample 2-91 | 0.93 | GG | TT | AA | GG | CC | GG | AA | AA |
| Sample 2-92 | 1.07 | GG | TT | AA | GG | CC | GG | AA | Not detected |
| Sample 2-93 | 1.13 | GG | TT | AA | GG | CC | GG | AA | AA |
| Sample 2-94 | 0.89 | GG | CT | AA | GG | CC | GG | AA | GA |
| Sample 2-95 | 1.04 | GG | CT | GA | GG | CC | GG | AA | AA |
| Sample 2-96 | 1.09 | GG | TT | AA | GG | CC | GG | AA | AA |
| Sample 2-97 | 4.02 | AA | CC | GG | AA | TT | AA | GG | GG |
| Sample 2-98 | 2.16 | AA | CC | GG | AA | CT | AA | GA | GG |

The significance analysis results show that the genotype of Scaffold4239:309117 is extremely significantly correlated with (+)-catechin content, the correlation coefficient is 0.7, p-value is 8.79×10⁻¹⁶, F-value (6.91/3.94) is 92.9, which is a recessive mutation, and the (+)-catechin content in the dry matter of tea soup corresponding to an AA genotype sample has extremely significant difference compared with GG and GA genotype samples. It is statistically judged that, when the genotype of the sample is double mutant AA, the (+)-catechin content in the dry matter in the tea plant is more likely to be higher than the normal average of the sample of which the genotype is wild type GG or single mutant GA.

The significance analysis results show that, the genotype of Scaffold3614: 66549 is extremely significantly correlated with (+)-catechin content, the correlation coefficient is 0.59, p-value is 1.24×10⁻¹⁰, F-value (6.91/3.94) is 52.1, which is a recessive mutation, the (+)-catechin content in the dry matter corresponding to a CC genotype sample has extremely significant difference compared with TT and CT genotype samples. It is statistically judged that, when the genotype of the sample is double mutant CC, the (+)-catechin content in the dry matter in the tea plant is more likely to be higher than the sample of which the genotype is wild type TT or single mutant CT.

The significance analysis results show that, the genotype of Scaffold349: 3413816 is extremely significantly correlated with (+)-catechin content, the correlation coefficient is 0.48, p-value is 4.78×10⁻⁷, F-value (6.91/3.94) is 29.2, which is a recessive mutation, the (+)-catechin content in the dry matter of tea soup corresponding to a GG genotype sample has extremely significant difference compared with GA and AA genotype samples. It is statistically judged that, when the genotype of the sample is double mutant GG, the (+)-catechin content in the dry matter in the tea plant is more likely to be higher than the sample of which the genotype is wild type AA or single mutant GA.

The significance analysis results show that, the genotype of Scaffold1989: 2316385 is extremely significantly correlated with (+)-catechin content, the correlation coefficient is 0.45, p-value is 3.16×10⁻⁶, F-value (6.91/3.94) is 18.7, which is a recessive mutation, the (+)-catechin content in the dry matter of tea soup corresponding to an AA genotype sample has extremely significant difference compared with GA and GG genotype samples. It is statistically judged that, when the genotype of the sample is double mutant AA, the (+)-catechin content in the dry matter in the tea plant is more likely to be higher than the normal average of the sample of which the genotype is wild type GG or single mutant GA.

The significance analysis results show that, the genotype of Scaffold451: 940283 is extremely significantly correlated with (+)-catechin content, the correlation coefficient is 0.54, p-value is 8.76×10⁻¹⁶, F-value (6.91/3.94) is 92.9, which is a recessive mutation, the (+)-catechin content in the dry matter of tea soup corresponding to a TT genotype sample has extremely significant difference compared with CC and CT genotype samples. It is statistically judged that, when the genotype of the sample is double mutant TT, the (+)-catechin content in the dry matter in the tea plant is more likely to be higher than the normal average of the sample of which the genotype is wild type CC or single mutant CT.

The significance analysis results show that, the genotype of Scaffold3727:442660 is extremely significantly correlated with (+)-catechin content, the correlation coefficient is 0.64, p-value is 1.60×10⁻¹², F-value (6.91/3.94) is 65.9, which is a recessive mutation, the (+)-catechin content in the dry matter of tea soup corresponding to an AA genotype sample has extremely significant difference compared with GG and GA genotype samples. It is statistically judged that, when the genotype of the sample is double mutant AA, the (+)-catechin content in the dry matter in the tea plant is more likely to be higher than the normal average of the sample of which the genotype is wild type GG or single mutant GA.

The significance analysis results show that, the genotype of Scaffold115: 803980 is extremely significantly correlated with (+)-catechin content, the correlation coefficient is 0.70, p-value is 8.79×10⁻¹⁶, F-value (6.91/3.94) is 92.95, which is a recessive mutation, the (+)-catechin content in the dry matter of the tea plant corresponding to a GG genotype sample has extremely significant difference compared with AA and GA genotype samples, it is statistically judged that, when the genotype of the sample is double mutant GG, the (+)-catechin content in the dry matter in the tea plant is more likely to be higher than the sample of which the genotype is wild type AA or single mutant GA.

The significance analysis results show that, the genotype of Scaffold920: 281727 is extremely significantly correlated with (+)-catechin content, the correlation coefficient is 0.54, p-value is 1.19×10⁻⁸, F-value (6.91/3.94) is 38.92, which is a recessive mutation, the (+)-catechin content in the dry matter of the tea plant corresponding to a GG genotype sample has extremely significant difference compared with AA and GA genotype samples, it is statistically judged that, when the genotype of the sample is double mutant GG, the (+)-catechin content in the dry matter in the tea plant is more likely to be higher than the sample of which the genotype is wild type AA or single mutant GA.

### Embodiment 3 Kit for Evaluating Tea Plant (+)-Catechin Content

### I. Composition

The primers for the SNP site 1 which have the nucleotide sequences shown as SEQ ID NO: 2 and SEQ ID NO: 3, the primers for the SNP site 2 which have the nucleotide sequences shown as SEQ ID NO: 5 and SEQ ID NO: 6, the primers for the SNP site 3 which have the nucleotide sequences shown as SEQ ID NO: 8 and SEQ ID NO: 9, the primers for the SNP site 4 which have the nucleotide sequences shown as SEQ ID NO: 11 and SEQ ID NO: 12, the primers for the SNP site 5 which have the nucleotide sequences shown as SEQ ID NO: 14 and SEQ ID NO: 15, the primers for the SNP site 6 which have the nucleotide sequences shown as SEQ ID NO: 17 and SEQ ID NO: 18, the primers for the SNP site 7 which have the nucleotide sequences shown as SEQ ID NO: 20 and SEQ ID NO: 21, and/or the primers for the SNP site 8 which have the nucleotide sequences shown as SEQ ID NO: 23 and SEQ ID NO: 24, 2×Taq PCR Master Mix, ddH₂O.

In particular, primer F for SNP site 1: GAAGACTAACCCGTATCGAG (SEQ ID NO: 2);
primer R for SNP site 1: ACACTTACAGTCTCTTGCGG (SEQ ID NO: 3);
primer F for SNP site 2: GATGACACAACCCTCATCTG (SEQ ID NO: 5);
primer R for SNP site 2: AATGTATGCCCGGTAAGGAC (SEQ ID NO: 6);
primer F for SNP site 3: TCTCTGCACTGTTGTCACTC (SEQ ID NO: 8);
primer R for SNP site 3: CACCACACTTTCTTAGAAGG (SEQ ID NO: 9);
primer F for SNP site 4: GATTTGACCTTCAACGTGGG (SEQ ID NO: 11);
primer R for SNP site 4: TGCAGCGTTTGTGTTTGCAG (SEQ ID NO: 12);
primer F for SNP site 5: GTAATAGACGGTGCAAACCC (SEQ ID NO: 14);
primer R for SNP site 5: CAAAGTATTTGGGAGCGCTG (SEQ ID NO: 15);
primer F for SNP site 6: TTGTCCGTGTCCAATCCTTG (SEQ ID NO: 17);
primer R for SNP site 6: ATTGACCACCTGGAAGAAGC (SEQ ID NO: 18);
primer F for SNP site 7: CTTCATCTCCACCACACTTC (SEQ ID NO: 20);
primer R for SNP site 7: GCCCAAAGTAGCAAAGAGAG (SEQ ID NO: 21);
primer F for SNP site 8: TTCGCATTCGTCCTTTTGGG (SEQ ID NO: 23);
primer R for SNP site 8: ACGTGCTACATTCTCCATCC (SEQ ID NO: 24).

### II. Usage Method

(1) The CTAB method was used to extract total DNA from buds of tea plant, it was ensured that A260/A280 of each DNA sample was between 1.8 and 2.0, and the concentration was greater than 100 μg/μl;

### (2) PCR Amplification

Detection primers with nucleotide sequences shown as SEQ ID NO: 2 and SEQ ID NO: 3, SEQ ID NO: 5 and SEQ ID NO: 6, SEQ ID NO: 8 and SEQ ID NO: 9, SEQ ID NO: 11 and SEQ ID NO: 12, SEQ ID NO: 14 and SEQ ID NO: 15, SEQ ID NO: 17 and SEQ ID NO: 18, SEQ ID NO: 20 and SEQ ID NO: 21, and SEQ ID NO: 23 and SEQ ID NO: 24 were used for detecting SNP site 1, SNP site 2, SNP site 3, SNP site 4, SNP site 5, SNP site 6, SNP site 7 and SNP site 8, respectively.

| | |
|---|---|
| 2 × Taq PCR | 5 μl |
| Master Mix primers | Each 0.5 μl |
| DNA template | 1 μl |
| ddH₂O | 3 μl |

PCR amplification procedure was as follows:

| | | |
|---|---|---|
| 95° C. | 5 minutes | |
| 95° C. | 30 seconds | ×45 cycles |
| 56° C. | 30 seconds | |
| 72° C. | 30 seconds | |
| 72° C. | 2 minutes | |
| 4° C. | forever | |

### (3) Product Purification

The PCR amplification products were subjected to gel electrophoresis, followed by recovery and purification using a commercially available gel electrophoresis DNA recovery kit.

A band with a fragment length of about 240 bp in the amplification product of the primers shown in SEQ ID NO: 2 and SEQ ID NO: 3 was selected for recovery and purification.

A band with a fragment length of about 240 bp in the amplification product of the primers shown in SEQ ID NO: 5 and SEQ ID NO: 6 was selected for recovery and purification.

A band with a fragment length of about 240 bp in the amplification product of the primers shown in SEQ ID NO: 8 and SEQ ID NO: 9 was selected for recovery and purification.

A band with a fragment length of about 240 bp in the amplification product of the primers shown in SEQ ID NO: 11 and SEQ ID NO: 12 was selected for recovery and purification.

A band with a fragment length of about 240 bp in the amplification product of the primers shown in SEQ ID NO: 14 and SEQ ID NO: 15 was selected for recovery and purification.

A band with a fragment length of about 240 bp in the amplification product of the primers shown in SEQ ID NO: 17 and SEQ ID NO: 18 was selected for recovery and purification.

A band with a fragment length of about 240 bp in the amplification product of the primers shown in SEQ ID NO: 20 and SEQ ID NO: 21 was selected for recovery and purification.

A band with a fragment length of about 240 bp in the amplification product of the primers shown in SEQ ID NO: 23 and SEQ ID NO: 24 was selected for recovery and purification.

### (4) Sequencing and Interpretation of Results

The amplification products of the primers shown in SEQ ID NO: 2 and SEQ ID NO: 3 were recovered and purified and sent to a sequencing company for Sanger sequencing. The sequencing results were compared with the nucleotide sequence shown in SEQ ID NO: 1. According to FIG. 2 (bold and underlined parts denote upstream and downstream primers), the site Scaffold4239:309117 is located at the 73rd base of the amplification product. It is statistically judged that, when the genotype of the sample is double mutant AA, the (+)-catechin content in the dry matter in the tea plant is more likely to be higher than the normal average of the sample of which the genotype is wild type GG or single mutant GA.

The amplification products of the primers shown in SEQ ID NO: 5 and SEQ ID NO: 6 were recovered and purified and sent to a sequencing company for Sanger sequencing. The sequencing results were compared with the nucleotide sequence shown in SEQ ID NO: 4. According to FIG. 3 (bold and underlined parts denote upstream and downstream primers), the site Scaffold3614: 66549 is located at the 137th base of the amplification product. It is statistically judged that, when the genotype of the sample is double mutant CC, the (+)-catechin content in the dry matter in the tea plant is more likely to be higher than the sample of which the genotype is wild type TT or single mutant CT.

The amplification products of the primers shown in SEQ ID NO: 8 and SEQ ID NO: 9 were recovered and purified and sent to a sequencing company for Sanger sequencing. The sequencing results were compared with the nucleotide sequence shown in SEQ ID NO: 7. According to FIG. 4 (bold and underlined parts denote upstream and downstream primers), the site Scaffold349: 3413816 is located at the 160th base of the amplification product. It is statistically judged that, when the genotype of the sample is double mutant GG, the (+)-catechin content in the dry matter in the tea plant is more likely to be higher than the sample of which the genotype is wild type AA or single mutant GA.

The amplification products of the primers shown in SEQ ID NO: 11 and SEQ ID NO: 12 were recovered and purified and sent to a sequencing company for Sanger sequencing. The sequencing results were compared with the nucleotide sequence shown in SEQ ID NO: 10. According to FIG. 5 (bold and underlined parts denote upstream and downstream primers), the site Scaffold1989: 2316385 is located at the 175th base of the amplification product. It is statistically judged that, when the genotype of the sample is double mutant AA, the (+)-catechin content in the dry matter in the tea plant is more likely to be higher than the normal average of the sample of which the genotype is wild type GG or single mutant GA.

The amplification products of the primers shown in SEQ ID NO: 14 and SEQ ID NO: 15 were recovered and purified and sent to a sequencing company for Sanger sequencing. The sequencing results were compared with the nucleotide sequence shown in SEQ ID NO: 13. According to FIG. 6 (bold and underlined parts denote upstream and downstream primers), the site Scaffold451: 940283 is located at the 161st base of the amplification product. It is statistically judged that, when the genotype of the sample is double mutant TT, the (+)-catechin content in the dry matter in the tea plant is more likely to be higher than the normal average of the sample of which the genotype is wild type CC or single mutant CT.

The amplification products of the primers shown in SEQ ID NO: 17 and SEQ ID NO: 18 were recovered and purified and sent to a sequencing company for Sanger sequencing. The sequencing results were compared with the nucleotide sequence shown in SEQ ID NO: 16. According to FIG. 7 (bold and underlined parts denote upstream and downstream primers), the site Scaffold3727:442660 is located at the 197th base of the amplification product. It is statistically judged that, when the genotype of the sample is double mutant AA, the (+)-catechin content in the dry matter in the tea plant is more likely to be higher than the normal average of the sample of which the genotype is wild type GG or single mutant GA.

The amplification products of the primers shown in SEQ ID NO: 20 and SEQ ID NO: 21 were recovered and purified and sent to a sequencing company for Sanger sequencing. The sequencing results were compared with the nucleotide sequence shown in SEQ ID NO: 19. According to FIG. 8 (bold and underlined parts denote upstream and downstream primers), the site Scaffold115: 803980 is located at the 164th base of the amplification product. It is statistically judged that, when the genotype of the sample is double mutant GG, the (+)-catechin content in the dry matter in the tea plant is more likely to be higher than the sample of which the genotype is wild type AA or single mutant GA.

The amplification products of the primers shown in SEQ ID NO: 23 and SEQ ID NO: 24 were recovered and purified and sent to a sequencing company for Sanger sequencing. The sequencing results were compared with the nucleotide sequence shown in SEQ ID NO: 22. According to FIG. 9 (bold and underlined parts denote upstream and downstream primers), the site Scaffold920: 281727 is located at the 106th base of the amplification product. It is statistically judged that, when the genotype of the sample is double mutant GG, the (+)-catechin content in the dry matter in the tea plant is more likely to be higher than the sample of which the genotype is wild type AA or single mutant GA.

### Embodiment 4 Use of Kit for Evaluating Tea Plant (+)-Catechin Content

### I. Experimental Method

The kit in Embodiment 3 was used to detect 98 tea plant samples in Embodiment 2.

### II. Experiment Results

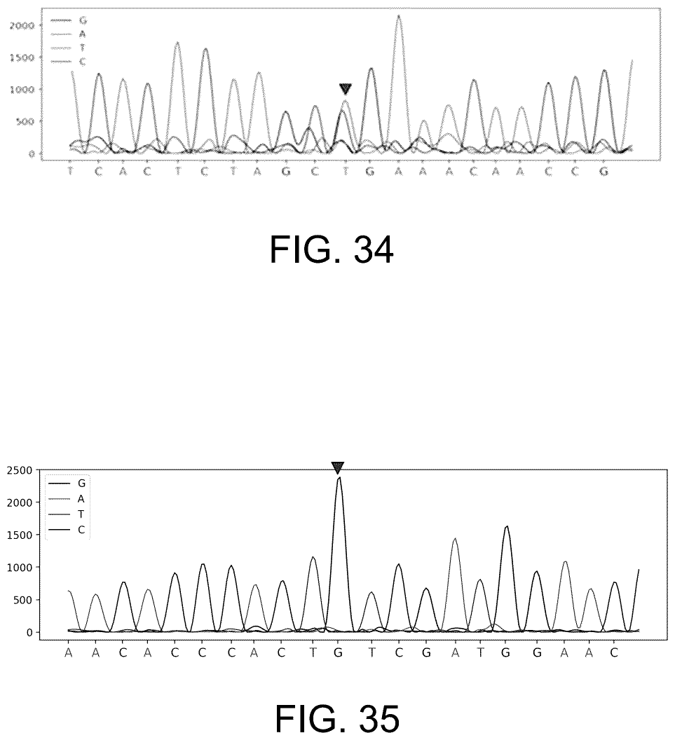

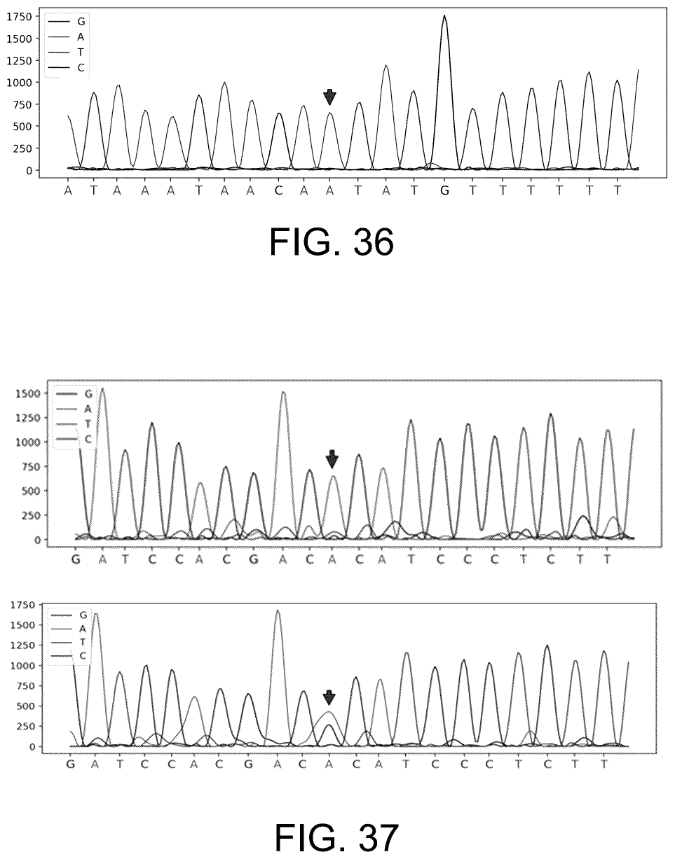

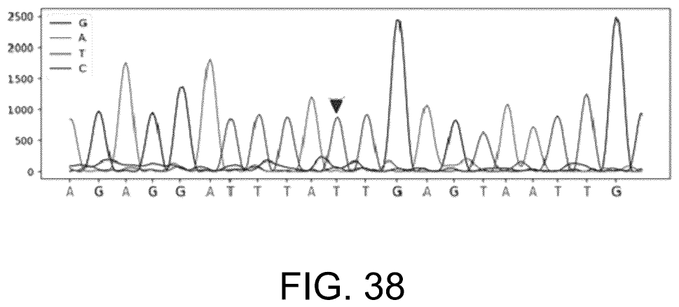

The detection results are consistent with those of Embodiment 2 using the SnapShot technology platform. This kit can be used to evaluate the tea plant (+)-catechin content. The sequencing peaks of some samples are shown in FIG. 34 to FIG. 38.

What is claimed is:

1. A method for evaluating tea plant (+)-catechin content, comprising using a pair of primers of a molecular marker for detecting and evaluating the tea plant (+)-catechin content, the primers have nucleotide sequences shown as SEQ ID NO:17 and SEQ ID NO:18, and the molecular marker is located at a SNP site of tea genomes Scaffold3727:442660, which is the 501st base of SEQ ID NO:16.
